(19) United States
(12) Patent Application Publication
(10) Pub. No.: US 2023/0207049 A1
(43) Pub. Date: Jun. 29, 2023

(54) DETERMINING PATHOGENIC RFC1 EXPANSIONS FROM SEQUENCING DATA

(71) Applicant: Illumina, Inc., San Diego, CA (US)

(72) Inventors: Sai Chen, San Diego, CA (US); Michael A. Eberle, San Diego, CA (US)

(21) Appl. No.: 17/837,642

(22) Filed: Jun. 10, 2022

Related U.S. Application Data
(60) Provisional application No. 63/209,608, filed on Jun. 11, 2021.

(51) Int. Cl. G16B 20/10; G16B 20/20; G16B 30/10; G16B 5/20

(52) U.S. Cl. CPC G16B 20/10; G16B 5/20; G16B 20/20; G16B 30/10

(57) ABSTRACT

Disclosed herein include systems, devices, and methods for determining repeat expansion status (e.g., pathogenic, carrier, and benign) of a locus of a gene of interest (e.g., at, or at about, chr4:39348424 of hg38 for RFC1). After aligning sequence reads to a sequence graph, the number of occurrences of repeat sequences satisfying predetermined criteria and the frequency of a pathogenic repeat sequence can be determined, which are in turn used to determine a repeat expansion status.

### RELATED APPLICATIONS

The present application claims priority under 35 U.S.C. § 119(e) to U.S. Provisional Application No. 63/209,608, filed Jun. 11, 2021. The content of the related application is incorporated herein by reference in its entirety.

### REFERENCE TO SEQUENCE LISTING

The present application is being filed along with a Sequence Listing in electronic format. The Sequence Listing is provided as a file entitled Sequence_Listing_47CX-311978-US.txt, created on May 29, 2022, which is 10 kilobytes in size. The information in the electronic format of the Sequence Listing is incorporated herein by reference in its entirety.

### BACKGROUND

### Field

This disclosure relates generally to the field of processing sequence data, and more particularly to determining repeats.

### Background

A biallelic intronic AAGGG repeat expansion in the replication factor C subunit (RFC1) gene can cause familial cerebellar ataxia, neuropathy, and vestibular areflexia syndrome (CANVAS) and late-onset ataxia. Current diagnosis methods of this pathogenic AAGGG expansion are time consuming and cannot be performed in large-scale. One method is clinical whole genome sequencing (cWGS) screening by manual examination of reads from alignment. However, in this high GC genomic region, the base quality of pair-end reads drops, which makes manual examination more difficult and error-prone, especially when there are pathogenic AAGGGs or other high-GC repeat patterns. There is a need for an automated and accurate method to identify this pathogenic expansion from sequencing data, including cWGS data.

### SUMMARY

Disclosed herein include methods for determining a replication factor C subunit 1 (RFC1) repeat expansion status. In some embodiments, a method for determining RFC1 repeat expansion status is under control of a processor (such as a hardware processor or a virtual processor) and comprises: (a) receiving a plurality of sequence reads generated from a sample obtained from a subject. The method can comprise: (b) aligning the plurality of sequence reads to a sequence graph to generate a plurality of aligned sequence reads. The sequence graph can represent a locus of RFC1. The sequence graph can comprise a repeat sequence representation flanked by non-repeat sequences of the locus of RFC1. The plurality of aligned sequence reads can comprise the plurality of sequence reads and alignments of the plurality of sequence reads to the sequence graph. The method can comprise: (c) determining a number of occurrences of a plurality of repeat sequences in aligned sequence reads of the plurality of aligned sequence reads using a first occurrence threshold and a first quality threshold. The method can comprise: (d) determining a frequency indication of a number of occurrences of a pathogenic repeat sequence relative to a total number of occurrences of the plurality of repeat sequences. The method can comprise: (e) determining a status of a repeat expansion at the locus of RFC1 of the subject using the frequency indication of the number of occurrences of the pathogenic repeat sequence relative to the total number of occurrences of the plurality of repeat sequences.

In some embodiments, the method comprises: determining the subject has zero, one, or two alleles with a repeat expansion at the locus of RFC1 using the plurality of aligned sequence reads. In some embodiments, the repeat expansion at the locus of RFC1 is at about chr4:39348424 of hg38, or a corresponding position of another reference genome sequence.

In some embodiments, the status of the repeat expansion at the locus of RFC1 is a pathogenic status, a carrier status, or a benign status. In some embodiments, the repeat expansion is associated with or causes a disease. The disease can be cerebellar ataxia, neuropathy, and vestibular areflexia syndrome (CANVAS). In some embodiments, the method comprises: confirming the status of the repeat expansion at the locus of RFC1 of the subject using one or more diagnosis methods. The one or more diagnosis methods can comprise polymerase chain reaction (PCR) and Sanger sequencing, southern blots, and linkage analysis.

In some embodiments, the subject has two alleles with repeat expansion at the locus of RFC1. In some embodiments, determining the status of the repeat expansion at the locus of RFC1 comprises: determining the frequency indication of the number of occurrences of the pathogenic repeat sequence relative to the total number of occurrences of the plurality of repeat sequences is greater than a first status threshold. Determining the status of the repeat expansion at the locus of RFC1 can comprise: determining the status of the repeat expansion at the locus of RFC1 as pathogenic status.

In some embodiments, determining the status of the repeat expansion at the locus of RFC1 comprises: determining the frequency indication of the number of occurrences of the pathogenic repeat sequence relative to the total number of occurrences of the plurality of repeat sequences is less than or equal to a first status threshold and greater than or equal to a second status threshold. Determining the status of the repeat expansion at the locus of RFC1 can comprises: determining the status of the repeat expansion at the locus of RFC1 as carrier status.

In some embodiments, determining the status of the repeat expansion at the locus of RFC1 comprises: determining the frequency indication of the number of occurrences of the pathogenic repeat sequence relative to the total number of occurrences of the plurality of repeat sequences is less than or equal to a first status threshold and greater than or equal to a second status threshold. Determining the status of the repeat expansion at the locus of RFC1 comprises can comprise: determining a frequency indication of a number of occurrences of the pathogenic repeat sequence and a sequence with a high sequence similarity to the pathogenic repeat sequence is greater than a third status threshold. For example, the pathogenic repeat sequence and the sequence similar to the pathogenic repeat sequence can differ by one base. Determining the status of the repeat expansion at the locus of RFC1 can comprise: determining a frequency indication of a number of occurrences of the pathogenic sequence is greater than a fourth status threshold. Determining the status of the repeat expansion at the locus of RFC1 can comprise: determining the status of the repeat expansion at the locus of RFC1 as carrier status.

In some embodiments, determining the status of the repeat expansion at the locus of RFC1 comprises: determining the frequency indication of the number of occurrences of the pathogenic repeat sequence relative to the total number of occurrences of the plurality of repeat sequences is less than a second status threshold. Determining the status of the repeat expansion at the locus of RFC1 can comprise: determining the status of the repeat expansion at the locus of RFC1 as benign status.

In some embodiments, the subject has one allele of RFC1 with repeat expansion at the locus of RFC1. In some embodiments, determining the number of occurrences of the plurality of repeat sequences in the aligned sequence reads of the plurality of sequence reads comprises: determining the number of occurrences of the plurality of repeat sequences in the aligned sequence reads of the plurality of aligned sequence reads that are from the one allele of RFC1 with repeat expansion at the locus of RFC1. Determining the status of the repeat expansion at the locus of RFC1 can comprise: determining the frequency indication of the number of occurrences of the pathogenic repeat sequence relative to the total number of occurrences of the plurality of repeat sequences is greater than a first status threshold. Determining the status of the repeat expansion at the locus of RFC1 can comprise: determining the status of the repeat expansion at the locus of RFC1 as pathogenic status.

In some embodiments, determining the number of occurrences of the plurality of repeat sequences in the aligned sequence reads of the plurality of sequence reads comprises: determining the number of occurrences of the plurality of repeat sequences in the aligned sequence reads of the plurality of aligned sequence reads that are from the one allele of RFC1 with repeat expansion at the locus of RFC1. Determining the status of the repeat expansion at the locus of RFC1 can comprise: determining the frequency indication of the number of occurrences of the pathogenic repeat sequence relative to the total number of occurrences of the plurality of repeat sequences is less than or equal to a first status threshold. Determining the status of the repeat expansion at the locus of RFC1 can comprise: determining the status of the repeat expansion at the locus of RFC1 as carrier status.

In some embodiments, the method comprises: selecting the aligned sequence reads that are from the one allele of RFC1 with repeat expansion at the locus of RFC1 based on the alignments of the aligned sequence reads. In some embodiments, the aligned sequence reads that are from the one allele of RFC1 with repeat expansion at the locus of RFC1 comprise the aligned sequence reads that are (i) in-repeat reads or (ii) flanking reads each with an overlap to the repeat expansion at the locus of RFC1 greater than a repeat expansion overlap threshold. The repeat expansion overlap threshold can be about 60 base pairs. The aligned sequence reads that are from the one allele of RFC1 with repeat expansion at the locus of RFC1 can comprise (i) all the aligned sequence reads that are in-repeat reads and (ii) not all of the aligned sequence reads that are flanking reads.

In some embodiments, the subject has zero allele with repeat expansion at the locus of RFC1. The status of the repeat expansion at the locus of RFC1 can be benign status.

In some embodiments, the repeat expansion at the locus of RFC1 comprises greater than a threshold total copies of one or more repeat sequences. The threshold total copies of one or more repeat sequences can be 20 total copies of one or more repeat sequences. In some embodiments, each of the plurality of repeat sequences has a number of occurrences greater than or equal to the first occurrence threshold with each occurrence having a number of bases each having a quality score greater than or equal to the first quality threshold. The first occurrence threshold can be 2. The first quality threshold can be about 20. The number of bases of the repeat sequence each having a quality score greater than the first quality threshold can be 5. In some embodiments, each of the occurrences has a number of bases each having a quality score greater than or equal to a second quality threshold. The second quality threshold can be about 20. The number of bases of each of the occurrences having a quality score greater than the second quality threshold can be 3.

In some embodiments, the repeat sequence representation is degenerate. The repeat sequence representation can be AARRG. The repeat sequence representation can be at least 5 bases in length. The repeat sequence representation can be 5 bases in length. The repeat sequence representation can be 6 bases in length. Each of the plurality of repeat sequences can be at least 5 bases in length. Each of the plurality of repeat sequences can be 5 bases in length. Each of the plurality of repeat sequences can be 6 bases in length. The pathogenic repeat sequence can be AAGGG or ACAGG. The pathogenic repeat sequence can have a GC content of at least 60%.

In some embodiments, the frequency indication of the number of occurrences of the pathogenic repeat sequence relative to the total number of occurrences of the plurality of repeat sequences is a percentage of the number of occurrences of the pathogenic repeat sequence out of the total number of occurrences of the plurality of repeat sequences. The frequency indication of the number of occurrences of the pathogenic repeat sequence relative to the total number of occurrences of the plurality of repeat sequences can be a ratio of the number of occurrences of the pathogenic repeat sequence over the total number of occurrences of the plurality of repeat sequences.

In some embodiments, the plurality of sequence reads is aligned to the locus of RFC1. In some embodiments, receiving the plurality of sequence reads generated from the sample obtained comprises: aligning a second plurality of sequence reads comprising the plurality of sequence reads to a reference genome sequence. Receiving the plurality of sequence reads generated from the sample obtained can comprise: selecting the plurality of sequence reads from the second plurality of sequence reads, wherein the plurality of sequence reads is aligned to the locus of RFC1.

In some embodiments, the plurality of sequence reads comprises sequence reads that are about 100 base pairs to about 1000 base pairs in length each. The plurality of sequence reads can comprise paired-end sequence reads. The plurality of sequence reads can comprise single-end sequence reads. The plurality of sequence reads can be generated by targeted sequencing and/or whole genome sequencing (WGS), optionally wherein the WGS is clinical WGS (cWGS). In some embodiments, the sample comprises cells, cell-free DNA, cell-free fetal DNA, amniotic fluid, a blood sample, a biopsy sample, or a combination thereof. In some embodiments, the reference genome sequence comprises a reference human genome sequence. The subject can be a human subject.

Disclosed herein include systems for determining a repeat expansion status of a gene of interest. In some embodiments, a system for determining repeat expansion status of a gene of interest comprising: non-transitory memory configured to store executable instructions; and a processor (e.g., a hardware processor or a virtual processor) in communication with the non-transitory memory. The processor can be programmed by the executable instructions to perform: (a) receiving a plurality of sequence reads generated from a sample obtained from a subject. The processor can be programmed by the executable instructions to perform: (b) aligning the plurality of sequence reads to a sequence graph to generate a plurality of aligned sequence reads. The sequence graph can represent a locus of a gene of interest. The sequence graph can comprise a repeat sequence representation flanked by non-repeat sequences of the locus of the gene. The plurality of aligned sequence reads can comprise the plurality of sequence reads. The plurality of aligned sequence reads can comprise alignments of the plurality of sequence reads to the sequence graph. The processor can be programmed by the executable instructions to perform: (c) determining a number of occurrences of a plurality of repeat sequences in aligned sequence reads of the plurality of aligned sequence reads using a first occurrence threshold and a first quality threshold. The processor can be programmed by the executable instructions to perform: (d) determining a frequency indication of a number of occurrences of a pathogenic repeat sequence relative to a total number of occurrences of the plurality of repeat sequence. The processor can be programmed by the executable instructions to perform: (e) determining a status of a repeat expansion at the locus of the gene of interest of the subject using the frequency indication of the number of occurrences of the pathogenic repeat sequence relative to the total number of occurrences of the plurality of repeat sequences.

In some embodiments, the gene of interest is replication factor C subunit 1 (RFC1). The locus of the gene of interest with the repeat expansion is at about chr4:39348424 of hg38, or a corresponding position of another reference genome sequence. The repeat expansion can be associated with or causes a disease. The disease can be cerebellar ataxia, neuropathy, and vestibular areflexia syndrome (CANVAS). The repeat sequence representation can be AARRG. The pathogenic repeat sequence can be AAGGG or ACAGG.

In some embodiments, the processor is programmed by the executable instructions to perform: determining the subject has zero, one, or two alleles with a repeat expansion at the locus of the gene of interest using the plurality of aligned sequence reads. In some embodiments, the status of the repeat expansion at the locus of the gene of interest is a pathogenic status, a carrier status, or a benign status. The repeat expansion can be associated with or causes a disease. The disease can be a neurologic disease. In some embodiments, the processor is programmed by the executable instructions to perform: receiving conformation of the status of the repeat expansion at the locus of the gene of interest of the subject determined using one or more diagnosis systems. The one or more diagnosis systems can comprise polymerase chain reaction (PCR) and Sanger sequencing, southern blots, and linkage analysis.

In some embodiments, the subject has two alleles with repeat expansion at the locus of the gene of interest. In some embodiments, determining the status of the repeat expansion at the locus of the gene of interest comprises: determining the frequency indication of the number of occurrences of the pathogenic repeat sequence relative to the total number of occurrences of the plurality of repeat sequences is greater than a first status threshold. Determining the status of the repeat expansion at the locus of the gene of interest can comprise: determining the status of the repeat expansion at the locus of the gene of interest as pathogenic status.

In some embodiments, determining the status of the repeat expansion at the locus of the gene of interest comprises: determining the frequency indication of the number of occurrences of the pathogenic repeat sequence relative to the total number of occurrences of the plurality of repeat sequences is less than or equal to a first status threshold and greater than or equal to a second status threshold. Determining the status of the repeat expansion at the locus of the gene of interest can comprise: determining the status of the repeat expansion at the locus of the gene of interest as carrier status.

In some embodiments, determining the status of the repeat expansion at the locus of the gene of interest comprises: determining the frequency indication of the number of occurrences of the pathogenic repeat sequence relative to the total number of occurrences of the plurality of repeat sequences is less than or equal to a first status threshold and greater than or equal to a second status threshold. Determining the status of the repeat expansion at the locus of the gene of interest can comprise: determining a frequency indication of a number of occurrences of the pathogenic repeat sequence and a sequence with a high sequence similarity to the pathogenic repeat sequence is greater than a third status threshold. For example, the pathogenic repeat sequence and the sequence similar to the pathogenic repeat sequence differs by one base. Determining the status of the repeat expansion at the locus of the gene of interest can comprise: determining a frequency indication of a number of occurrences of the pathogenic sequence is greater than a fourth status threshold Determining the status of the repeat expansion at the locus of the gene of interest can comprise: determining the status of the repeat expansion at the locus of the gene of interest as carrier status.

In some embodiments, determining the status of the repeat expansion at the locus of the gene of interest comprises: determining the frequency indication of the number of occurrences of the pathogenic repeat sequence relative to the total number of occurrences of the plurality of repeat sequences is less than a second status threshold. Determining the status of the repeat expansion at the locus of the gene of interest can comprise: determining the status of the repeat expansion at the locus of the gene of interest as benign status.

In some embodiments, the subject has one allele of the gene of interest with repeat expansion at the locus of the gene of interest. In some embodiments, determining the number of occurrences of the plurality of repeat sequences in the aligned sequence reads of the plurality of sequence reads comprises: determining the number of occurrences of the plurality of repeat sequences in the aligned sequence reads of the plurality of aligned sequence reads that are from the one allele of the gene of interest with repeat expansion at the locus of the gene of interest. Determining the status of the repeat expansion at the locus of the gene of interest can comprise: determining the frequency indication of the number of occurrences of the pathogenic repeat sequence relative to the total number of occurrences of the plurality of repeat sequences is greater than a first status threshold. Determining the status of the repeat expansion at the locus of the gene of interest can comprise: determining the status of the repeat expansion at the locus of the gene of interest as pathogenic status.

In some embodiments, wherein determining the number of occurrences of the plurality of repeat sequences in the aligned sequence reads of the plurality of sequence reads comprise: determining the number of occurrences of the plurality of repeat sequences in the aligned sequence reads of the plurality of aligned sequence reads that are from the one allele of the gene of interest with repeat expansion at the locus of the gene of interest. Determining the status of the repeat expansion at the locus of the gene of interest can comprise: determining the frequency indication of the number of occurrences of the pathogenic repeat sequence relative to the total number of occurrences of the plurality of repeat sequences is less than or equal to a first status threshold. Determining the status of the repeat expansion at the locus of the gene of interest can comprise: determining the status of the repeat expansion at the locus of the gene of interest as carrier status.

In some embodiments, the processor is programmed by the executable instructions to perform: selecting the aligned sequence reads that are from the one allele of the gene of interest with repeat expansion at the locus of the gene of interest based on the alignments of the aligned sequence reads. In some embodiments, the aligned sequence reads that are from the one allele of the gene of interest with repeat expansion at the locus of the gene of interest comprise the aligned sequence reads that are (i) in-repeat reads or (ii) flanking reads each with an overlap to the repeat expansion at the locus of the gene of interest greater than a repeat expansion overlap threshold, optionally wherein the repeat expansion overlap threshold is about 60 base pairs. The aligned sequence reads that are from the one allele of the gene of interest with repeat expansion at the locus of the gene of interest can comprise (i) all the aligned sequence reads that are in-repeat reads and (ii) not all of the aligned sequence reads that are flanking reads.

In some embodiments, the subject has zero allele with repeat expansion at the locus of the gene of interest. The status of the repeat expansion at the locus of the gene of interest can be benign status.

In some embodiments, the repeat expansion at the locus of the gene of interest comprises greater than a threshold total copies of one or more repeat sequences. The threshold total copies of one or more repeat sequences can be 20 total copies of one or more repeat sequences. In some embodiments, each of the plurality of repeat sequences has a number of occurrences greater than or equal to the first occurrence threshold with each occurrence having a number of bases each having a quality score greater than or equal to the first quality threshold. The first occurrence threshold can be 2. The first quality threshold can be about 20. The number of bases of the repeat sequence each having a quality score greater than the first quality threshold can be 5. In some embodiments, each of the occurrences has a number of bases each having a quality score greater than or equal to a second quality threshold. The second quality threshold can be about 20. The number of bases of each of the occurrences having a quality score greater than the second quality threshold can be 3.

In some embodiments, the repeat sequence representation is degenerate. The repeat sequence representation and/or each of the plurality of repeat sequences can be at least 5 bases

in length. The repeat sequence representation and/or each of the plurality of repeat sequences can be 5 bases in length. The repeat sequence representation and/or each of the plurality of repeat sequences can be 6 bases in length. In some embodiments, the pathogenic repeat sequence has a GC content of at least 60%.

In some embodiments, wherein the frequency indication of the number of occurrences of the pathogenic repeat sequence relative to the total number of occurrences of the plurality of repeat sequences is a percentage of the number of occurrences of the pathogenic repeat sequence out of the total number of occurrences of the plurality of repeat sequences or a ratio of the number of occurrences of the pathogenic repeat sequence over the total number of occurrences of the plurality of repeat sequences.

In some embodiments, the plurality of sequence reads is aligned to the locus of the gene of interest. In some embodiments, receiving the plurality of sequence reads generated from the sample obtained comprises: aligning a second plurality of sequence reads comprising the plurality of sequence reads to a reference genome sequence. Receiving the plurality of sequence reads generated from the sample obtained can comprise: selecting the plurality of sequence reads from the second plurality of sequence reads, wherein the plurality of sequence reads is aligned to the locus of the gene of interest.

In some embodiments, the plurality of sequence reads comprises sequence reads that are about 100 base pairs to about 1000 base pairs in length each. The plurality of sequence reads comprises paired-end sequence reads. The plurality of sequence reads comprises single-end sequence reads. In some embodiments, the plurality of sequence reads is generated by targeted sequencing and/or whole genome sequencing (WGS), optionally wherein the WGS is clinical WGS (cWGS). In some embodiments, the sample comprises cells, cell-free DNA, cell-free fetal DNA, amniotic fluid, a blood sample, a biopsy sample, or a combination thereof. In some embodiments, the reference genome sequence comprises a reference human genome sequence. The subject can be a human subject.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 shows a non-limiting exemplary schematic illustration of a sequence graph representing RFC1 locus and categorization of reads that overlap with repeats as spanning reads, in-repeat reads, and flanking reads.

FIG. 2 shows a non-limiting exemplary visualization of reads of a patient sample that overlap with the repeat. The patient was confirmed by PCR to have two expanded AAGGG haplotypes.

FIG. 3 shows a non-limiting exemplary visualization of reads of a patient sample that overlap with the repeat. The human patient was a presumed patient. However, PCR did not validate the presumption. PCR suggested AAGAG repeats instead of AAGGG repeats.

FIGS. 4A-4B show schematic illustrations of non-limiting exemplary 5-mer filtering and counting.

FIGS. 5A-5B illustrate that carriers need a better separation for the two haplotypes.

FIGS. 6A-6B are non-limiting exemplary schematic illustrations of sequence reads that come from the expanded allele.

FIG. 7 shows the percentage of 5-mer repeats in the expanded allele for samples with one short allele and one expanded allele in a cohort of 75 patients.

FIG. 8 shows the percentage of 5-mer repeats in the expanded allele for samples with one short allele and one expanded allele for a Polaris population of 150 unrelated healthy individuals.

FIG. 9 is a flow diagram showing an exemplary method of determining an RFC1 repeat expansion status.

FIG. 10 is a flow diagram showing an exemplary method of determining a repeat expansion status of a gene of interest (e.g., RFC1).

FIG. 11 is a block diagram of an illustrative computing system configured to implement determining a repeat expansion status of a gene of interest (e.g., RFC1).

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for determining a replication factor C subunit 1 (RFC1) repeat expansion status. In some embodiments, a method for determining RFC1 repeat expansion status is under control of a processor (such as a hardware processor or a virtual processor) and comprises: (a) receiving a plurality of sequence reads generated from a sample obtained from a subject. The method can comprise: (b) aligning the plurality of sequence reads to a sequence graph to generate a plurality of aligned sequence reads. The sequence graph can represent a locus of RFC1. The sequence graph can comprise a repeat sequence representation flanked by non-repeat sequences of the locus of RFC1. The plurality of aligned sequence reads can comprise the plurality of sequence reads and alignments of the plurality of sequence reads to the sequence graph. The method can comprise: (c) determining a number of occurrences of a plurality of repeat sequences in aligned sequence reads of the plurality of aligned sequence reads using a first occurrence threshold and a first quality threshold. The method can comprise: (d) determining a frequency indication of a number of occurrences of a pathogenic repeat sequence relative to a total number of occurrences of the plurality of repeat sequences. The method can comprise: (e) determining a status of a repeat expansion at the locus of RFC1 of the subject using the frequency indication of the number of occurrences of the pathogenic repeat sequence relative to the total number of occurrences of the plurality of repeat sequences.

Disclosed herein include systems for determining a repeat expansion status of a gene of interest. In some embodiments, a system for determining repeat expansion status of a gene of interest comprising: non-transitory memory configured to store executable instructions; and a processor (such as a hardware processor or a virtual processor) in communication with the non-transitory memory. The processor can be programmed by the executable instructions to perform: (a) receiving a plurality of sequence reads generated from a sample obtained from a subject. The processor can be programmed by the executable instructions to perform: (b) aligning the plurality of sequence reads to a sequence graph to generate a plurality of aligned sequence reads. The sequence graph can represent a locus of a gene of interest. The sequence graph can comprise a repeat sequence representation flanked by non-repeat sequences of the locus of the gene. The plurality of aligned sequence reads can comprise the plurality of sequence reads. The plurality of aligned sequence reads can comprise alignments of the plurality of sequence reads to the sequence graph. The processor can be programmed by the executable instructions to perform: (c) determining a number of occurrences of a plurality of repeat sequences in aligned sequence reads of the plurality of aligned sequence reads using a first occurrence threshold and a first quality threshold. The processor can be programmed by the executable instructions to perform: (d) determining a frequency indication of a number of occurrences of a pathogenic repeat sequence relative to a total number of occurrences of the plurality of repeat sequence. The processor can be programmed by the executable instructions to perform: (e) determining a status of a repeat expansion at the locus of the gene of interest of the subject using the frequency indication of the number of occurrences of the pathogenic repeat sequence relative to the total number of occurrences of the plurality of repeat sequences.

### Determining Pathogenic RFC1 Expansion

A biallelic intronic AAGGG repeat expansion in the replication factor C subunit (RFC1) gene is recently discovered as the cause of familial cerebellar ataxia, neuropathy, and vestibular areflexia syndrome (CANVAS) and a frequent cause of late-onset ataxia. In the general population, this repeat expansion is very divergent with many different benign repeat patterns. Current diagnosis methods for this pathogenic AAGGG expansion include polymerase chain reaction (PCR) and Sanger sequencing, Southern blots and linkage analysis, but all of these methods are time consuming and cannot be performed in large-scale. Compared to these methods, clinical whole genome sequencing (cWGS) screening makes it possible for this expansion to be identified in a massive parallel way. However, there is no automated and accurate way to identify this pathogenic expansion from cWGS data. Currently, this pathogenic expansion is identified by manual examination of reads from alignment, such as alignment by ExpansionHunter (Dolzhenko, E. et al. ExpansionHunter Denovo: a computational method for locating known and novel repeat expansions in short-read sequencing data. Genome Biol 21, 102 (2020). https://doi.org/10.1186/s13059-020-02017-z, the content of which is incorporated herein by reference in its entirety). When the sample size is large, this manual screening becomes extremely time-consuming.

Furthermore, in this high GC genomic region, the base quality of pair-end reads drops. This quality drop is correlated with what the repeat pattern is. This makes manual screening even more difficult and error-prone, especially when there are pathogenic AAGGGs or other high-GC repeat patterns. Because of that, there was an argument that it is not possible to accurately diagnose this pathogenic repeat from pair-end cWGS data.

Disclosed herein are methods to automatically and accurately screen this pathogenic expansion from sequencing data, such as cWGS data. There is no other method to screen this pathogenic repeat from sequencing data. The method implements a logic described herein to minimize the artifact that comes from decreased data quality in this region. For example, for one cWGS sample, a prior method determined more than 80 different repeat patterns and the percentage of AAGGG was only about 64%. In comparison, for the same sample, the method of the present disclosure determined only a few different repeat patterns and the percentage of AAGGG was more than 90%. The method implements rules to separate real pathogenic repeats from benign ones. The method can quickly screen a large population and output patients with double expanded AAGGG repeats, carriers with expanded AAGGG repeats on one haplotype and benign repeats on the other haplotype, and normal people with no expanded AAGGG on either haplotype. On one 30x cWGS sample, the method took less than one second to output the screening result (after ExpansionHunter had been run).

Repeat expansions in RFC1 has been identified as a cause for CANVAS. In GRCh38, RFC1 has (AAAAG)₁₁ repeats in chr4:39348424-39348479. There can be many other benign expansion patterns, such as (AAAAG)ₑₓₚ and possibly (AAAGG)ₑₓₚ. Homozygous (AAGGG)ₑₓₚ and (ACAGG)ₑₓₚ have been identified as pathogenic. Diagnosis methods for pathogenic RFC1 expansions include PCR & Sanger sequencing, Southern blots, linkage analysis, and clinical whole-genome sequencing (cWGS) screening. PCR & Sanger sequencing utilizes different primers for mutants and wildtypes. However, long-range PCR is more error-prone in repetitive regions. Southern blots are more difficult than PCR. Southern blots can confirm the presence of biallelic large expansions. Southern blots do not tell what expansions they are. Regarding linkage analysis, family-based studies have identified a few linkage disequilibrium (LD) single nucleotide polymorphisms (SNPs) for mutants. However, a few pedigrees do not guarantee the SNPs are in LD with the pathogenic repeats.

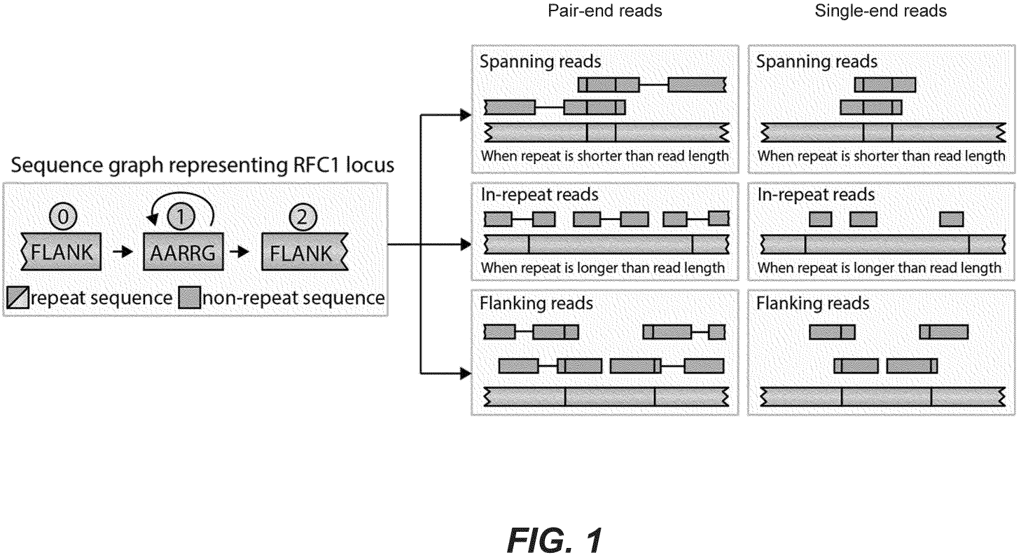

This pathogenic expansion can be identified with cWGS screening by manual examination of reads from, for example, ExpansionHunter (EH) alignment. The screening can be performed through ExpansionHunter, on a constructed repeat graph of (AARRG)ₙ, where R is A or G (FIG. 1), such that the reference AAAAG and pathogenic AAGGG can be genotyped simultaneously. In graph alignments, reads that overlap with repeats can be categorized as spanning reads, in-repeat reads, and flanking reads as illustrated in FIG. 1. A spanning read can occur when a repeat is shorter than the read length such that the read includes the repeat and the flanking regions on both end of the repeat. An in-repeat read can occur when the repeat is longer than the read length such that the entire read (whether a single-end sequencing read or a pair-end sequencing read) or one read of a pair-end sequencing read includes only a portion of the repeat and no flanking region of the repeat. A flanking read includes a portion of the repeat and the flanking region on one end of the repeat.

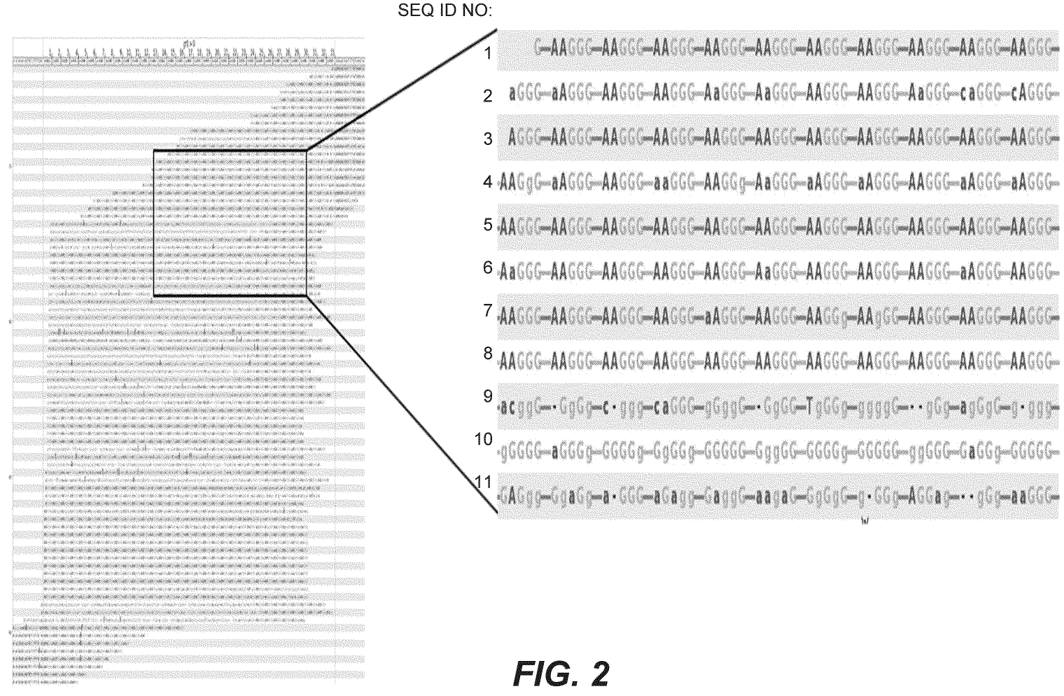

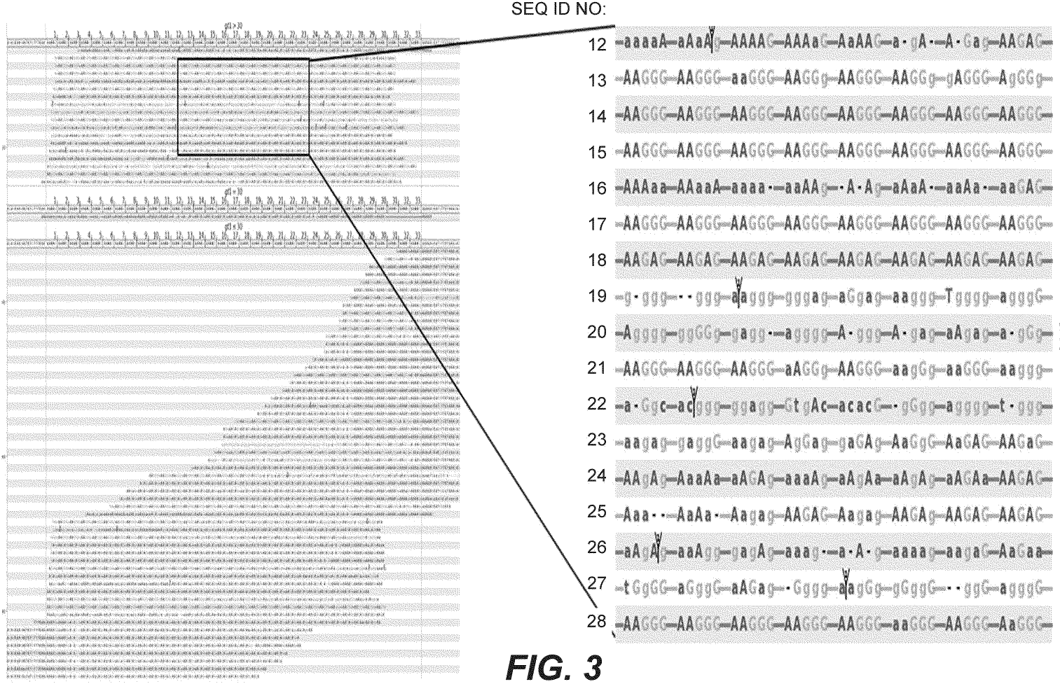

To identify pathogenic expansion with cWGS screening by manual examination, all reads that overlap with the repeats can be visualized through an EH visualization tool. The reads can be examined to see if the reads include pathogenic (AAGGG)ₙ. FIG. 2 shows the visualization of reads of a human patient sample that overlap with the repeat. The human patient FAM-092-D12 was confirmed by PCR to have two expanded AAGGG haplotypes. As shown in FIG. 2, the reads were either in-repeat reads and flanking reads mostly containing AAGGG. Because the repeat is longer than the read length, no spanning read was observed. However, manual examination can be difficult and inaccurate. For example, two different haplotypes can be difficult to differentiate. Patterns similar to pathogenic repeats (e.g., AAAGG) may result in false positives calls. FIG. 3 shows the visualization of reads of a patient sample that overlap with the repeat. The human patient FAM-062-F08 was a presumed patient. However, PCR did not validate the presumption. As illustrated in FIG. 3, it is difficult to tell what repeats they are from bare eyes. PCR suggested AAGAG repeats instead of AAGGG repeats.

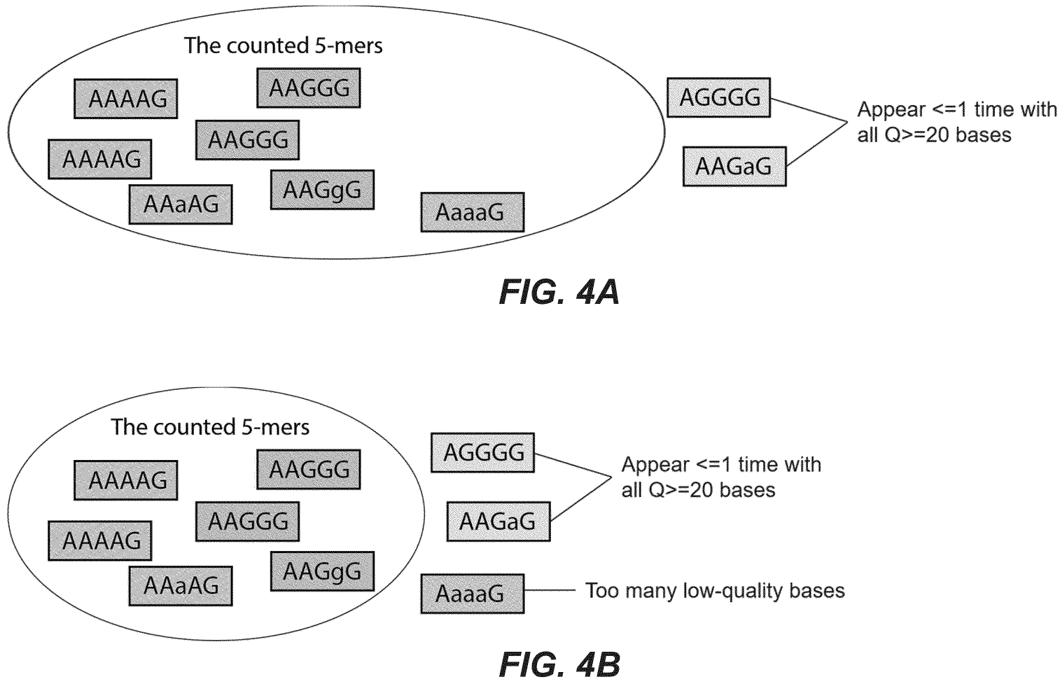

Disclosed herein are methods to automatically and accurately screen the pathogenic expansion from sequencing data, such as cWGS data. On graph-aligned reads, how many times each 5-mer was observed in the repeat-overlapping part can be counted. In some embodiments, to minimize the noise from decreased data quality, the following criterion can be used: Each type of 5-mer has two occurrences with all 5 bases having quality (Q) greater than or equal to a quality threshold (e.g., 20). Referring to FIG. 4A, a base with quality greater than or equal to 20 is shown capitalized. For example, of the four AAAAG/AAaAg/AaaaG 5-mers shown in FIG. 4A, two had all five bases with quality greater than or equal to 20 (the 5-mers shown as AAAAG), one had four bases with quality greater than or equal to 20 (the 5-mer shown as AAaAG), and one had two bases with quality greater than or equal to 20 (the 5-mer shown as AaaaG). The four AAAAG/AAaAg/AaaaG 5-mers satisfy this criterion and are counted. As another example, of the three AAGGG/AAGgG 5-mers shown in FIG. 4A, two had all five bases with quality greater than or equal to 20 (the 5-mers shown as AAGGG), and one had four bases with quality greater than or equal to 20 (the 5-mer shown as AAGgG). All three AAGGG/AAGgG 5-mers satisfy this criterion and are counted. As a further example, of the two remaining 5-mers shown in FIG. 4A, one had all five bases with quality greater than or equal to 20 (the 5-mer shown as AGGGG), and one had four bases with quality greater than or equal to 20 (the 5-mer shown as AAGaG). These two 5-mers do not satisfy the criterion that each type of 5-mer has two occurrences with all 5 bases having quality (Q) greater than or equal to a quality threshold of 20 because each type of these 5-mers has an occurrence of one. These two 5-mers are filtered and not counted.

In some embodiments, to minimize the noise from decreased data quality, the following criteria can be used: Each type of 5-mer has two occurrences with all 5 bases having quality (Q) greater than or equal to a quality threshold (e.g., 20), and each counted 5-mer has at least 3 bases having quality greater than or equal to a quality threshold (e.g., 20). Referring to FIG. 4B, a base with quality greater than or equal to 20 is shown capitalized. For example, of the four AAAAG/AAaAg/AaaaG 5-mers shown in FIG. 4B, two had all five bases with quality greater than or equal to 20 (the 5-mers shown as AAAAG), one had four bases with quality greater than or equal to 20 (the 5-mer shown as AAaAG), and one had two bases with quality greater than or equal to 20 (the 5-mer shown as AaaaG). The AAAAG/AAaAg 5-mers satisfy the criteria and are counted. The AaaaG 5-mers does not satisfy the criteria (because this 5-mer only has two bases with quality greater than or equal to 20) and are filtered and not counted. As another example, of the three AAGGG/AAGgG 5-mers shown in FIG. 4B, two had all five bases with quality greater than or equal to 20 (the 5-mers shown as AAGGG), and one had four bases with quality greater than or equal to 20 (the 5-mer shown as AAGgG). All three AAGGG/AAGgG 5-mers satisfy the criteria and are counted. As a further example, of the two remaining 5-mers shown in FIG. 4B, one had all five bases with quality greater than or equal to 20 (the 5-mer shown as AGGGG), and one had four bases with quality greater than or equal to 20 (the 5-mer shown as AAGaG). These two 5-mers do not satisfy the criterion that each type of 5-mer has two occurrences with all 5 bases having quality (Q) greater than or equal to a quality threshold of 20 because each type of these 5-mers had an occurrence of one) and are filtered and not counted.

Sequence reads of patient samples were processed and 5-mer counting was performed using the criteria. Real patients had almost 100% AAGGG (a pathogenic repeat). With the 5-mer filtering and counting rules, it was observed that 93% AAGGG in the PCR-validated patient FAM-062-F08 (See FIG. 2 and the accompanying description). The other 5-mers were AAAGG (2%) and GGGGG (5%). These other 5-mers are very likely to come from sequencing errors/artifacts.

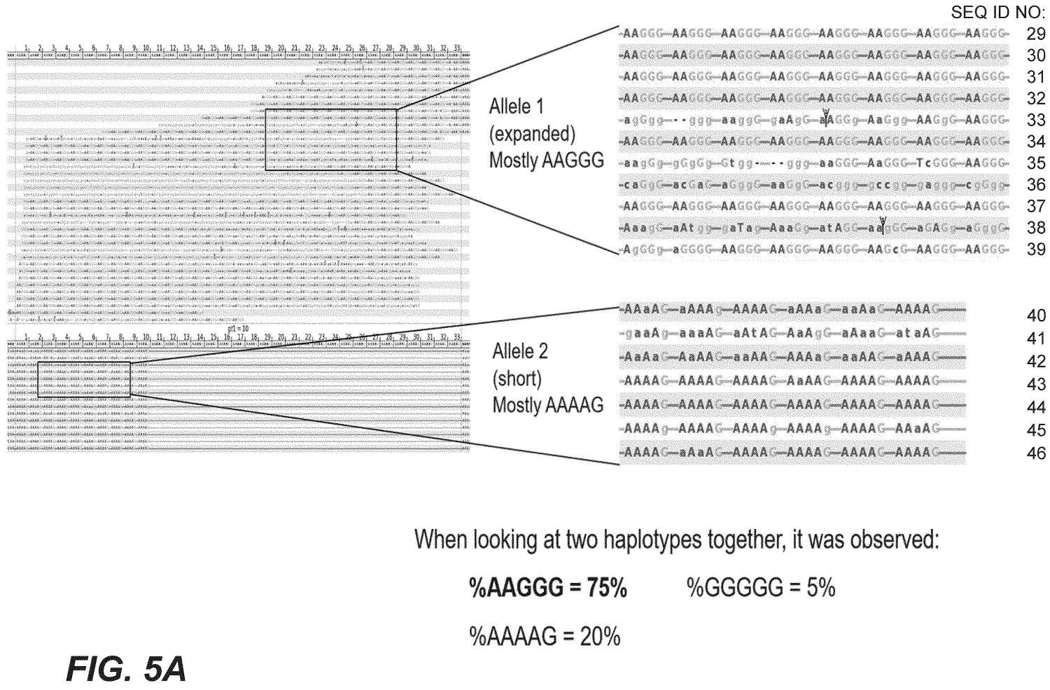

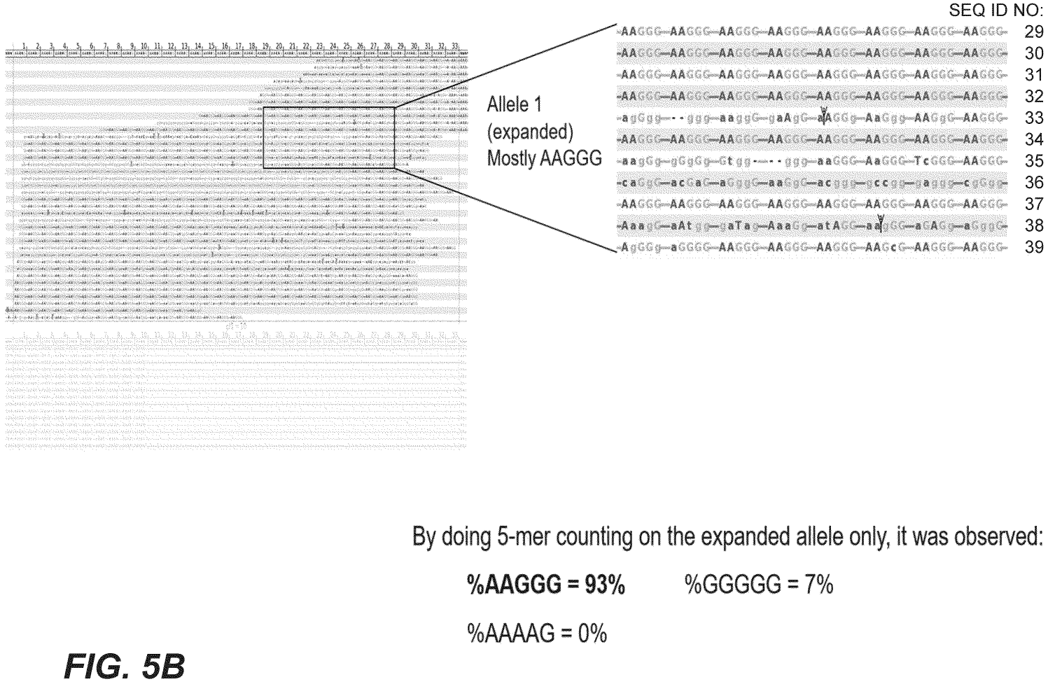

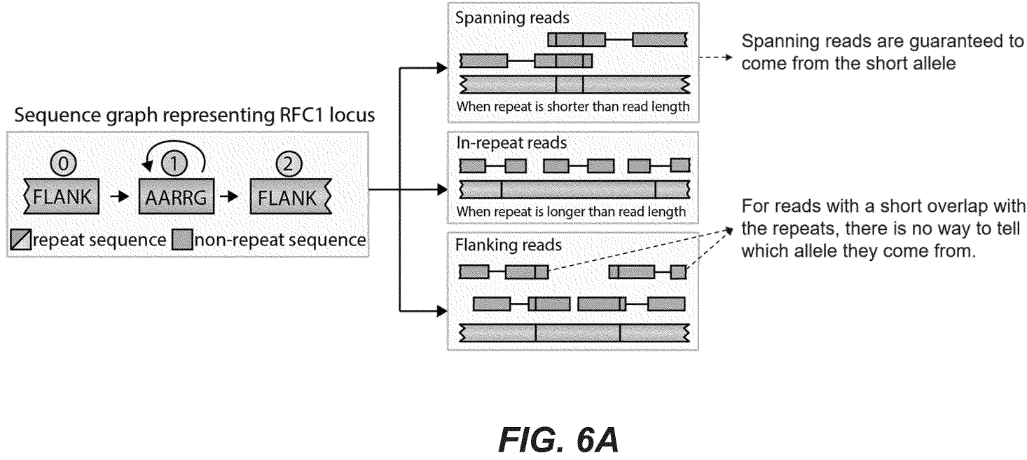

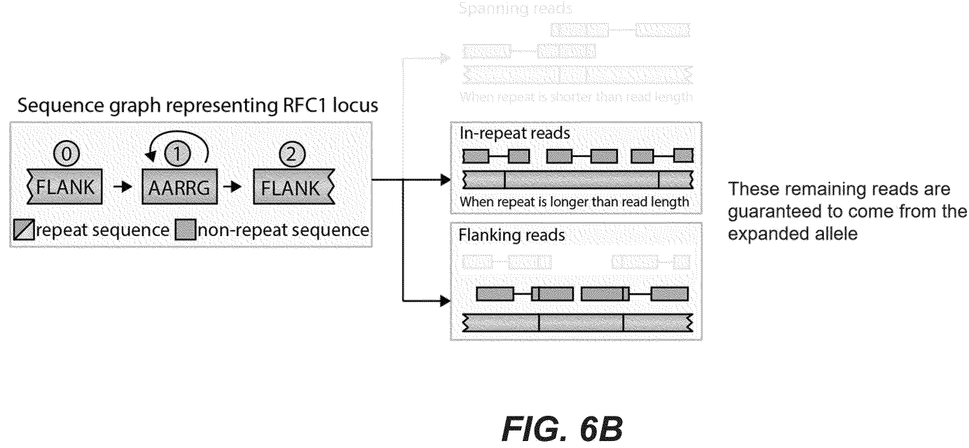

As illustrated in FIGS. 5A-5B, carriers need a better separation for the two haplotypes. In reads from the expanded allele, carriers had almost 100% AAGGG. For a patient who is a carrier with one expanded allele, sequence reads of the patient’s sample were processed and 5-mer filtering and counting using the criteria. With the 5-mer filtering the counting criteria, the patient was observed to have 75% AAGGG (FIG. 5A). By performing 5-mer filtering and counting on the reads from the expanded allele using the criteria, the patient was observed to have 93% AAGGG. Referring to FIGS. 6A-6B, spanning reads are from the short allele. For flanking reads with short overlaps with the repeats, it is uncertain which allele the reads come from. The remaining flanking reads shown in FIG. 6B and the in-repeat reads come from the expanded allele.

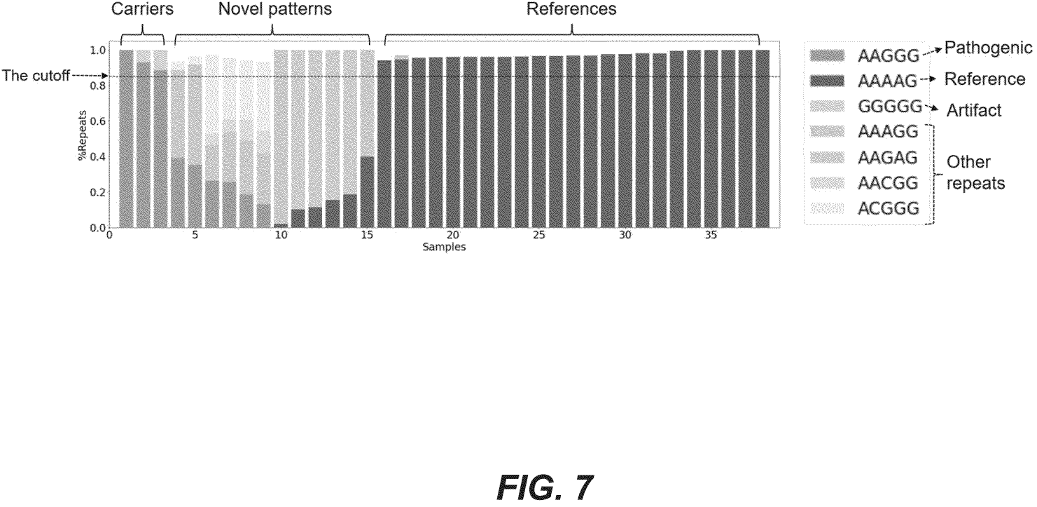

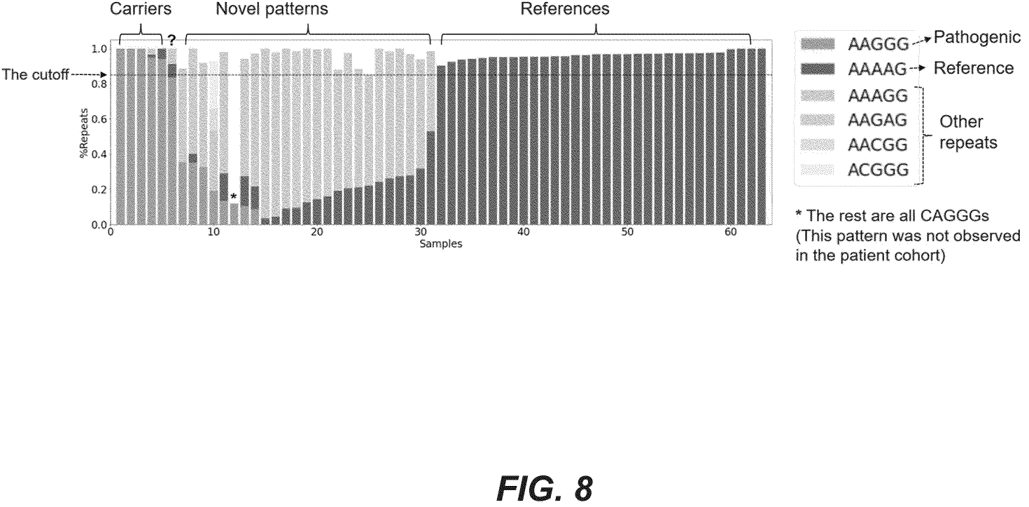

The samples of 77 patients with exhibiting signs of repeat expansions such as anticipation (earlier onset or more severe phenotype in subsequent generations) and/or neuropathy symptoms underwent cWGS. These patients were suspected to have some pathogenic RFC1 repeat expansions. None of them had any clinical documentation for known expansions. Failure to identify repeat expansions in these samples may be because of insufficient testing or associated with previously unknown pathogenic repeat expansions. With a general ExpansionHunter screening, RFC1 was identified as “expanded” in a few patients. Previously, real RFC1 patients were identified by manually looking at the sequence reads aligned to the repeat one by one. The methods of the present disclosure can be used for automatic screening of RFC1 status (pathogenic, carrier, or benign and/or the number of expansions such as zero, one, or two). Patterns of RFC1 expansions in the 77-patient cohort were examined. When screening the repeats in RFC1, greater than or equal to 85% for AAAAG was used for reference alleles, and greater than or equal to 85% for AAGGG was used for pathogenic alleles. Repeat patterns were identified by looking at the expanded allele. FIG. 7 shows the percentage of 5-mer repeats in the expanded allele for samples with one short allele and one expanded allele in the 75-patient cohort. Table 1 shows screening results on the patient cohort using the following criteria:
Pathogenic if two expanded alleles and greater than 85% AAGGG repeat.
Carrier if one expanded allele and greater than 85% AAGGG repeat, or if two expanded alleles and 30%-85% AAGGG repeat.
Benign if no expanded alleles, if one expanded allele and less than 85% AAGGG repeat, or if two expanded alleles and less than 30% AAGGG repeats.

TABLE 1

Screening results on 77-patient cohort

| | Total | # Pathogenic | # Carriers | # Benign |
|---|---|---|---|---|
| Two Expansions | 25 | 7* | 2 | 16 |
| One Expansion | 38 | - | 3 | 35 |
| Zero Expansions | 14 | - | - | 14 |

* Six samples have been validated by PCR. PCR validation has not been performed for one sample.

Patterns of RFC1 repeats in the Polaris population of 150 unrelated healthy individuals were examined. The Polaris population is a diverse population of Europeans, East Asians, and Africans. FIG. 8 shows the percentage of 5-mer repeats in the expanded allele for samples with one short allele and one expanded allele for this Polaris population. The analysis shows a high frequency for AAGGG and many other patterns. Table 1 shows screening results on the Polaris population using the following criteria:
Pathogenic if two expanded alleles and greater than 85% AAGGG repeat.
Carrier if one expanded allele and greater than 85% AAGGG repeat, or if two expanded alleles and 30%-85% AAGGG repeat.
Benign if no expanded alleles, if one expanded allele and less than 85% AAGGG repeat, or if two expanded alleles and less than 30% AAGGG repeats.

TABLE 2

Screening results on the Polaris population

| | Total | # Pathogenic | # Carriers | # Benign |
|---|---|---|---|---|
| Two Expansions | 24 | 0 | 3 | 21 |
| One Expansion | 63 | - | 5 | 58 |
| Zero Expansions | 63 | - | - | 63 |

### Determining RFC1 Repeat Expansion Status

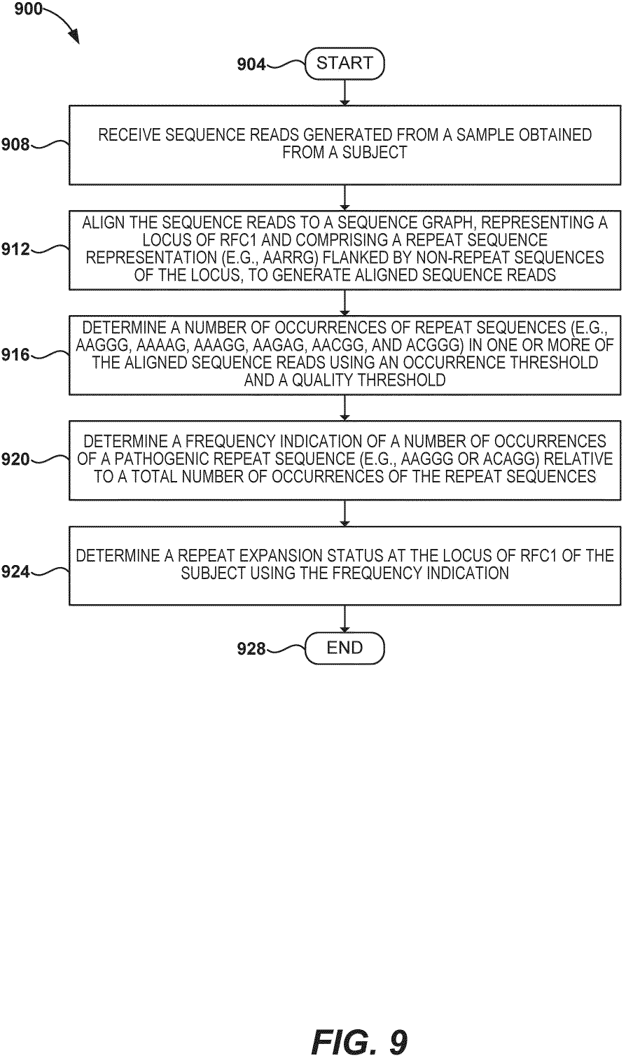

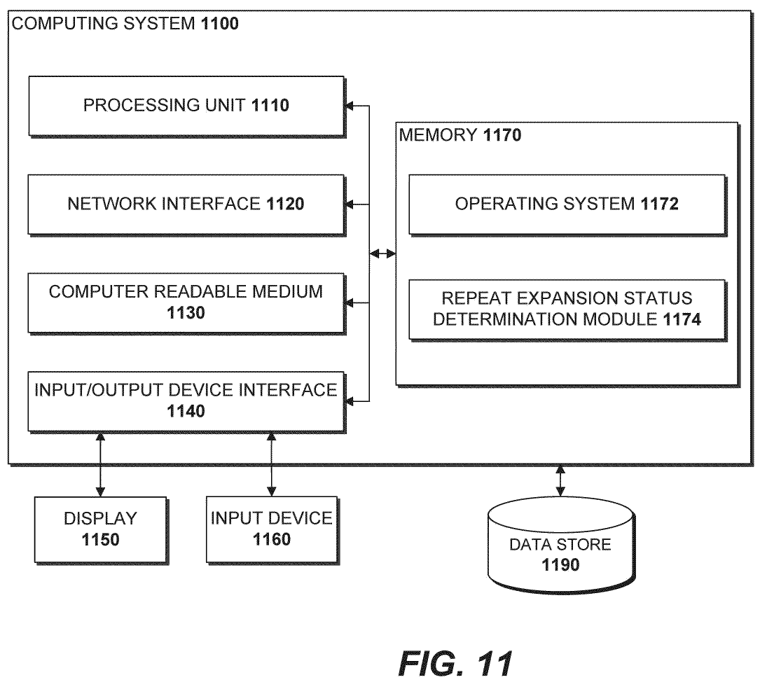

FIG. 9 is a flow diagram showing an exemplary method 900 of determining a replication factor C subunit 1 (RFC1) repeat expansion status. The method 900 may be embodied in a set of executable program instructions stored on a computer-readable medium, such as one or more disk drives, of a computing system. For example, the computing system 1100 shown in FIG. 11 and described in greater detail below can execute a set of executable program instructions to implement the method 900. When the method 900 is initiated, the executable program instructions can be loaded into memory, such as RAM, and executed by one or more processors of the computing system 1100. Although the method 900 is described with respect to the computing system 1100 shown in FIG. 11, the description is illustrative only and is not intended to be limiting. In some embodiments, the method 900 or portions thereof may be performed serially or in parallel by multiple computing systems.

After the method 900 begins at block 904, the method 900 proceeds to block 908, where a computing system (such as the computing system 1100 described with reference to FIG. 11) receives a plurality of sequence reads generated from a sample obtained from a subject. Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by targeted sequencing. The sequence reads can be generated by whole genome sequencing (WGS). The sequence reads can be generated by whole genome sequencing (WGS). The WGS can be clinical WGS (cWGS). The sample can comprise cells, cell-free DNA, cell-free fetal DNA, amniotic fluid, a blood sample, a biopsy sample, or a combination thereof. The subject can be a human subject.

The sequence reads received can include only reads from the locus of RFC1. For example, the plurality of sequence reads can be aligned to the locus of RFC1. Alternatively, the sequence reads received can include reads from the locus of RFC1 and elsewhere. The computing system can align a second plurality of sequence reads comprising the plurality of sequence reads aligned to the locus of RFC1 to a reference genome sequence. The reference genome sequence can comprise a reference human genome sequence, such as hg19 or hg38. The computing system can select the plurality of sequence reads that are aligned to the locus of RFC1 from the second plurality of sequence reads.

The computing system can store the sequence reads in memory. The computing system can load sequence reads into memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The method 900 proceeds from block 908 to block 912, where the computing system aligns the plurality of sequence reads to a sequence graph to generate a plurality of aligned sequence reads. The sequence graph can represent a locus of RFC1. The sequence graph can comprise a repeat sequence representation (e.g., AARRG where R is A or G) flanked by non-repeat sequences of the locus of RFC1. The plurality of aligned sequence reads can comprise or be associated with the plurality of sequence reads and alignments of the plurality of sequence reads to the sequence graph.

The method 900 proceeds from block 912 to block 916, where the computing system determines a number of occurrences of a plurality of repeat sequences (e.g., AAGGG, AAAAG, AAAGG, AAGAG, AACGG, and ACGGG) in aligned sequence reads of the plurality of aligned sequence reads using a first occurrence threshold (e.g., 2) and a first quality threshold (e.g., 20). Determining or counting the number of occurrences of the plurality of repeat sequences in the aligned sequence reads of the plurality of aligned sequence reads using the first occurrence threshold and the first quality threshold is referred to herein as 5-mer filtering and counting. The 5-mer filtering and counting can be for both alleles or for the expanded allele.

A repeat expansion at the locus of RFC1 can comprise greater than a threshold total copies (e.g., 20 total copies) of one or more repeat sequences. The threshold total copies of one or more repeat sequences can be, for example, 15, 16, 17, 18, 19, 20, 30, 40, 50, 60, 70, 80, 90, 100, or more or less, total copies of one or more repeat sequences.

In some embodiments, each of the plurality of repeat sequences has a number of occurrences greater than or equal to (or greater than) a first occurrence threshold with each occurrence having a number of bases each having a quality score greater than or equal to a first quality threshold. The first occurrence threshold can be, for example, 2, 3, 4, 5, 6, 7, 8, 9, 10, or more. The first quality threshold can be, for example, about 10, 15, 20, 25, 30, 35, 40, 45, 50, or more or less. The number of bases of the repeat sequence each having a quality score greater than (or greater than or equal to) the first quality threshold can be, for example, 4 or 5. For example, each type of 5-mer has two occurrences with all 5 bases having quality (Q) greater than or equal to a quality threshold (e.g., a first quality threshold) of 20. In some embodiments, each of the occurrences has a number of bases each having a quality score greater than or equal to (or a greater than) than a second quality threshold. The number of bases each having a quality score greater than or equal to (or greater than) the second quality threshold can be, for example, 2, 3, 4, or 5. The second quality threshold can be for example, about 10, 15, 20, 25, 30, 35, 40, 45, 50, or more or less. The number of bases of each of the occurrences having a quality score greater than the second quality threshold can be, for example, 2, 3, 4, or 5. For example, each counted 5-mer has at least 3 bases having quality greater than or equal to a quality threshold (e.g., a second quality threshold) of 20. In some embodiments, the first quality threshold and the second quality threshold are identical.

The repeat sequence representation can be degenerate. The repeat sequence representation can be AARRG, where R is A or G. The repeat sequence representation can be at least 5 bases in length. The repeat sequence representation can be 5 bases in length. The repeat sequence representation can be 6 bases in length. Each of the plurality of repeat sequences can be at least 5 bases in length. Each of the plurality of repeat sequences can be 5 bases in length. Each of the plurality of repeat sequences can be 6 bases in length. The repeat sequence representation and a repeat sequence can have an identical length.

The method 900 proceeds from block 916 to block 920, where the computing system determines a frequency indication of a number of occurrences of a pathogenic repeat sequence (e.g., AAGGG or ACAGG), or one or more pathogenic repeat sequences, relative to a total number of occurrences of the plurality of repeat sequences. The pathogenic repeat sequence can be AAGGG or ACAGG. The pathogenic repeat sequence can have a GC content of at least (or greater than) 50%, 55%, 60%, 65%, 70%, 75%, 80%, or more or less. The pathogenic repeat sequence can be at least 5 bases in length. The pathogenic repeat sequence can be 5 bases in length. The pathogenic repeat sequence can be 6 bases in length.

The frequency indication of the number of occurrences of the pathogenic repeat sequence relative to the total number of occurrences of the plurality of repeat sequences can be a percentage of the number of occurrences of the pathogenic repeat sequence out of the total number of occurrences of the plurality of repeat sequences. The frequency indication of the number of occurrences of the pathogenic repeat sequence relative to the total number of occurrences of the plurality of repeat sequences can be a ratio of the number of occurrences of the pathogenic repeat sequence over the total number of occurrences of the plurality of repeat sequences.

The method 900 proceeds from block 920 to block 924, where the computing system determines a status of a repeat expansion (or a repeat expansion status) at the locus of RFC1 of the subject using the frequency indication of the number of occurrences of the pathogenic repeat sequence relative to the total number of occurrences of the plurality of repeat sequences. The repeat expansion status at the locus of RFC1 can be a pathogenic status, a carrier status, or a benign status. The computing system can determine the subject has zero, one, or two alleles with a repeat expansion at the locus of RFC1 using the plurality of aligned sequence reads. The repeat expansion at the locus of RFC1 can be at or at about chr4:39348424 of hg38, or a corresponding position of another reference genome sequence. The repeat expansion can be associated with or cause a disease. The disease can be cerebellar ataxia, neuropathy, and vestibular areflexia syndrome (CANVAS) or ataxia.

Two expanded alleles. If both alleles are expanded, 5-mer counting of both expanded alleles can be performed. In some embodiments, the subject can have two alleles each with a repeat expansion at the locus of RFC1.

Two expanded alleles - Pathogenic status. To determine the repeat expansion status at the locus of RFC1, the computing system can determine the frequency indication of the number of occurrences of the pathogenic repeat sequence relative to the total number of occurrences of the plurality of repeat sequences is greater than (or greater than or equal to) a first status threshold. The first status threshold can be, for example, 60%, 65%, 70%, 75%, 80%, 85%, 90%, 95%, or more or less. The computing system can determine the repeat expansion status at the locus of RFC1 as the pathogenic status.

Two expanded alleles - Carrier status. To determine the repeat expansion status at the locus of RFC1, the computing system can determine the frequency indication of the number of occurrences of the pathogenic repeat sequence relative to the total number of occurrences of the plurality of repeat sequences is less than or equal to (or less than) a first status threshold and is greater than or equal to (or greater than) a second status threshold. The first status threshold can be, for example, 60%, 65%, 70%, 75%, 80%, 85%, 90%, 95%, or more or less. The second status threshold can be, for example, 10%, 15%, 20%, 25%, 30%, 35%, 40%, 45%, 50%, or more or less. The computing system can determine the repeat expansion status at the locus of RFC1 as the carrier status.

Two expanded alleles - Carrier status. To determine the repeat expansion status at the locus of RFC1, the computing system can determine the frequency indication of the number of occurrences of the pathogenic repeat sequence relative to the total number of occurrences of the plurality of repeat sequences is less than or equal to (or less than) a first status threshold and is greater than or equal to (or is greater than or equal to) a second status threshold. The first status threshold can be, for example, 60%, 65%, 70%, 75%, 80%, 85%, 90%, 95%, or more or less. The second status threshold can be, for example, 10%, 15%, 20%, 25%, 30%, 35%, 40%, 45%, 50%, or more or less.

The computing system can determine a frequency indication of a number of occurrences of (1) the pathogenic repeat sequence and (2) a sequence with a high sequence similarity to the pathogenic repeat sequence relative to the total number of occurrences of the plurality of repeat sequences. The sequence similarity of the pathogenic repeat sequence and the sequence with a high sequence similarity to the pathogenic repeat sequence can be, or be about, for example, 70%, 75%, 80%, 85%, 90%, 95%, or more or less. The pathogenic repeat sequence and the sequence similar to the pathogenic repeat sequence differs by one or more bases, such as 1, 2, 3, 4, 5, 6, 7, 8, 9, 10, or more, bases. The sequence with a high sequence similarity to the pathogenic repeat sequence may be non-pathogenic and/or associated with (e.g., linked with) benign expansion.

The frequency indication of the number of occurrences of (1) the pathogenic repeat sequence and (2) the sequence with a high sequence similarity to the pathogenic repeat sequence, relative to the total number of occurrences of the plurality of repeat sequences, can be a percentage of the number of occurrences of (1) the pathogenic repeat sequence and (2) the sequence with a high sequence similarity to the pathogenic repeat sequence out of the total number of occurrences of the plurality of repeat sequences. The frequency indication of the number of occurrences of (1) the pathogenic repeat sequence and (2) the sequence with a high sequence similarity to the pathogenic repeat sequence, relative to the total number of occurrences of the plurality of repeat sequences, can be a ratio of the number of occurrences of (1) the pathogenic repeat sequence and (2) the sequence with a high sequence similarity to the pathogenic repeat sequence over the total number of occurrences of the plurality of repeat sequences.

The computing system can determine frequency indication of a number of occurrences of the pathogenic repeat sequence and the sequence with a high sequence similarity to the pathogenic repeat sequence relative to the total number of occurrences of the plurality of repeat sequences is greater than (or greater than or equal to) a third status threshold. The third status threshold can be, for example, 70%, 75%, 80%, 85%, 90%, 95%, or more or less.

The computing system can determine the frequency indication of the number of occurrences of the pathogenic sequence is greater than (or greater than or equal to) a fourth status threshold. The fourth status threshold can be, for example, 5%, 10%, 15%, 20%, 25%, 30%, or more or less. Alternatively or additionally, the computing system can determine a frequency indication of a number of occurrences of the sequence with a high sequence similarity to the pathogenic sequence. The frequency indication of number of occurrences of the sequence with a high sequence similarity to the pathogenic repeat sequence, relative to the total number of occurrences of the plurality of repeat sequences, can be a percentage of the number of occurrences of the sequence with a high sequence similarity to the pathogenic repeat sequence out of the total number of occurrences of the plurality of repeat sequences. The frequency indication of the sequence with a high sequence similarity to the pathogenic repeat sequence, relative to the total number of occurrences of the plurality of repeat sequences, can be a ratio of the number of occurrences of the sequence with a high sequence similarity to the pathogenic repeat sequence over the total number of occurrences of the plurality of repeat sequences. The computing system can determine the frequency indication of the number of occurrences of the sequence with a high sequence similarity to the pathogenic sequence is less than or equal to (or less than) a fifth status threshold. The fifth status threshold can be, for example, 70%, 75%, 80%, 85%, 90%, 95%, or more or less. The computing system can determine the repeat expansion status at the locus of RFC1 as the carrier status.

In some embodiments, the computing system determines the frequency indication of the number of occurrences of the pathogenic sequence is less than or equal to (or less than) the fourth status threshold. Alternatively or additionally, the computing system can determine the frequency indication of the number of occurrences of the sequence with a high sequence similarity to the pathogenic sequence is greater than (or greater than or equal to) the fifth status threshold. The computing system can determine the repeat expansion status at the locus of RFC1 as the benign status.

For example, the pathogenic repeat sequence is AAGGG, and the sequence with a high sequence similarity to the pathogenic repeat sequence is AAAGG. The two sequences have a sequence identity of 80%. The two sequences differ by one base. The percentage of the AAGGG repeat sequence and the AAAGG repeat sequence out of all the repeat sequences can be greater than 80%. The percentage of the AAGGG repeat sequence out of all repeat sequences can be greater than 10%. Alternatively or additionally, the percentage of the AAAGG repeat sequence out of all the repeat sequences can be less than or equal to 90%. The computing system can determine the repeat expansion status at the locus of RFC1 as the carrier status. If the percentage of the AAGGG repeat sequence out of all repeat sequences is less than or equal to 10% and/or the percentage of the AAAGG repeat sequence out of all the repeat sequences is greater than 90%, the computing system can determine the repeat expansion status at the locus of RFC1 as the benign status.

Two expanded alleles - Benign status. To determine the repeat expansion status at the locus of RFC1, the computing system can determine the frequency indication of the number of occurrences of the pathogenic repeat sequence relative to the total number of occurrences of the plurality of repeat sequences is less than (or less than or equal to) a second status threshold. The second status threshold can be, for example, 10%, 15%, 20%, 25%, 30%, 35%, 40%, 45%, 50%, or more or less. The computing system can determine the repeat expansion status at the locus of RFC1 as the benign status.

One expanded allele. If one allele is expanded and one allele is not expanded, 5-mer counting of the expanded allele can be performed. In some embodiments, the subject has one allele of RFC1 with repeat expansion at the locus of RFC1.

One expanded allele - Pathogenic status. To determine the number of occurrences of the plurality of repeat sequences in the aligned sequence reads of the plurality of sequence reads, the computing system can determine the number of occurrences of the plurality of repeat sequences in the aligned sequence reads of the plurality of aligned sequence reads that are from the one allele of RFC1 with repeat expansion at the locus of RFC1. To determine the repeat expansion status at the locus of RFC1, the computing system can determine the frequency indication of the number of occurrences of the pathogenic repeat sequence relative to the total number of occurrences of the plurality of repeat sequences is greater than (or greater than or equal to) a first status threshold. The first status threshold can be, for example, 60%, 65%, 70%, 75%, 80%, 85%, 90%, 95%, or more or less. The computing system can determine the repeat expansion status at the locus of RFC1 as the pathogenic status.

One expanded allele - Carrier status. To determine the number of occurrences of the plurality of repeat sequences in the aligned sequence reads of the plurality of sequence reads, the computing system can determine the number of occurrences of the plurality of repeat sequences in the aligned sequence reads of the plurality of aligned sequence reads that are from the one allele of RFC1 with repeat expansion at the locus of RFC1. To determine the repeat expansion status at the locus of RFC1, the computing system can determine the frequency indication of the number of occurrences of the pathogenic repeat sequence relative to the total number of occurrences of the plurality of repeat sequences is less than or equal to (or less than) a first status threshold. The first status threshold can be, for example, 60%, 65%, 70%, 75%, 80%, 85%, 90%, 95%, or more or less. The computing system can determine the repeat expansion status at the locus of RFC1 as the carrier status.

To determine the repeat expansion status at the locus of RFC1 as the pathogenic status or the carrier status when there is one expanded allele, the computing system can select the aligned sequence reads that are from the one allele of RFC1 with repeat expansion at the locus of RFC1 based on the alignments of the aligned sequence reads. The aligned sequence reads that are from the one allele of RFC1 with repeat expansion at the locus of RFC1 can comprise the aligned sequence reads that are (i) in-repeat reads or (ii) flanking reads each with an overlap to the repeat expansion at the locus of RFC1 greater than a repeat expansion overlap threshold. The repeat expansion overlap threshold can be, for example, 30, 35, 40, 45, 50, 55, 60, 65, 70, 75, 80, 85, 90, or more or less, base pairs. The aligned sequence reads that are from the one allele of RFC1 with repeat expansion at the locus of RFC1 can comprise (i) all the aligned sequence reads that are in-repeat reads and (ii) not all of the aligned sequence reads that are flanking reads. An in-repeat read can occur when the repeat is longer than the read length such that the entire read (whether a single-end sequencing read or a pair-end sequencing read) or one read of a pair-end sequencing read includes only a portion of the repeat and no flanking region of the repeat. A flanking read includes a portion of the repeat and the flanking region on one end of the repeat.

No expanded allele. If both alleles are not expanded, 5-mer counting of both alleles is can be performed. In some embodiments, the subject has zero allele with repeat expansion at the locus of RFC1. The repeat expansion status at the locus of RFC1 can be benign status.

In some embodiments, the computing system can generate a user interface (UI), such as a graphical user interface, comprising or representing any results (including intermediate results) of the method 900. For example, the UI can comprise or represent the repeat expansion status. The UI can include, for example, a dashboard. The UI can include one or more UI elements. A UI element can comprise or represent the repeat expansion status. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion). In some embodiments, the computing system can generate a report comprising or representing any results (including intermediate results) of the method 900, such as the repeat expansion status.

The computing system can cause one or more diagnosis methods to be performed to confirm of the repeat expansion status at the locus of RFC1 of the subject. The UI or the report can comprise or indicate that one or more diagnosis methods should be performed to confirm the repeat expansion status at the locus of RFC1 of the subject. The computing system can receive confirmation of the repeat expansion status at the locus of RFC1 of the subject determined using one or more diagnosis methods. The one or more diagnosis methods can comprise polymerase chain reaction (PCR) and Sanger sequencing, southern blots, and linkage analysis.

In some embodiments, any threshold of the method 900 (such as an occurrence threshold (e.g., a first occurrence threshold), a quality threshold (e.g., a first quality threshold, or a second quality threshold), a threshold total copies, a status threshold (e.g., a first status threshold, a second status threshold, a third status threshold, a fourth status threshold, or a fifth status threshold), or a repeat expansion overlap threshold) can be determined using a number of samples, such as 100, 200, 300, 400, 500, 1000, 2000, 3000, 4000, 5000, or more or less, samples.

The method 900 ends at block 928.

### Determining Repeat Expansion Status of a Gene of Interest

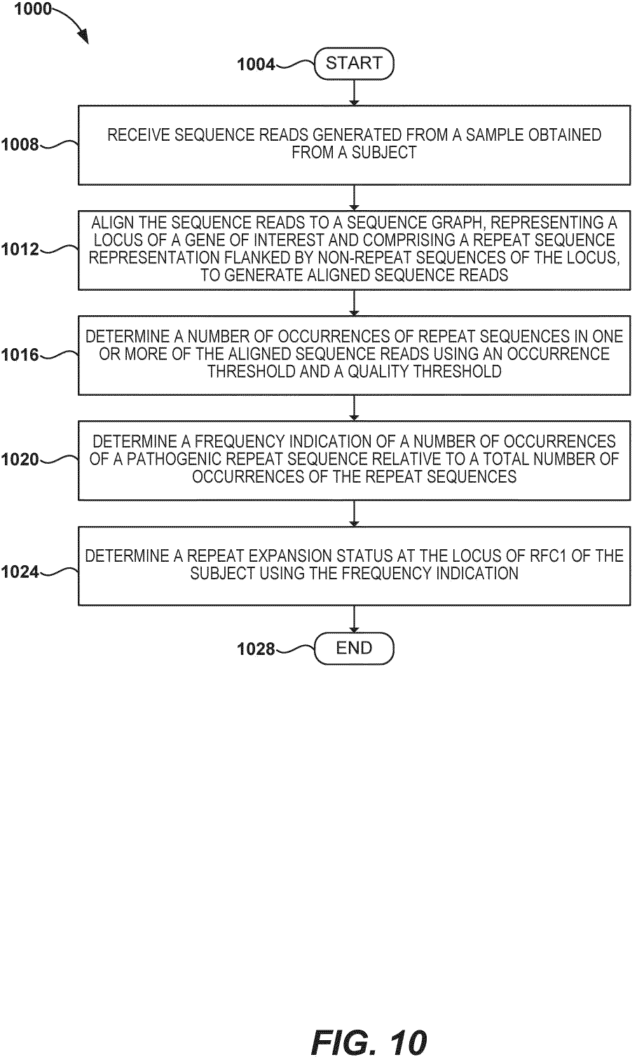

FIG. 10 is a flow diagram showing an exemplary method 1000 of determining a status of a repeat expansion (also referred to herein as repeat expansion status) of a gene of interest. The method 1000 may be embodied in a set of executable program instructions stored on a computer-readable medium, such as one or more disk drives, of a computing system. For example, the computing system 1100 shown in FIG. 11 and described in greater detail below can execute a set of executable program instructions to implement the method 1000. When the method 1000 is initiated, the executable program instructions can be loaded into memory, such as RAM, and executed by one or more processors of the computing system 1100. Although the method 1000 is described with respect to the computing system 1100 shown in FIG. 11, the description is illustrative only and is not intended to be limiting. In some embodiments, the method 1000 or portions thereof may be performed serially or in parallel by multiple computing systems.

After the method 1000 begins at block 1004, the method 1000 proceeds to block 1008, where a computing system (such as the computing system 1100 described with reference to FIG. 11) receives a plurality of sequence reads generated from a sample obtained from a subject. Sequence reads can be, for example, 50, 60, 70, 80, 90, 100, 110, 120, 130, 140, 150, 160, 170, 180, 190, 200, 300, 400, 500, 600, 700, 800, 900, 1000, 1250, 1500, 1750, 2000, or more base pairs (bps) in length each. For example, sequence reads are about 100 base pairs to about 1000 base pairs in length each. The sequence reads can comprise paired-end sequence reads. The sequence reads can comprise single-end sequence reads. The sequence reads can be generated by targeted sequencing. The sequence reads can be generated by whole genome sequencing (WGS). The sequence reads can be generated by whole genome sequencing (WGS). The WGS can be clinical WGS (cWGS). The sample can comprise cells, cell-free DNA, cell-free fetal DNA, amniotic fluid, a blood sample, a biopsy sample, or a combination thereof. The subject can be a human subject.

The sequence reads received can include only reads from the locus of the gene of interest. For example, the plurality of sequence reads can be aligned to the locus of the gene of interest. Alternatively, the sequence reads received can include reads from the locus of the gene of interest and elsewhere. The computing system can align a second plurality of sequence reads comprising the plurality of sequence reads aligned to the locus of the gene of interest to a reference genome sequence. The reference genome sequence can comprise a reference human genome sequence, such as hg19 or hg38. The computing system can select the plurality of sequence reads that are aligned to the locus of the gene of interest from the second plurality of sequence reads.

The computing system can store the sequence reads in memory. The computing system can load sequence reads into memory. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The method 1000 proceeds from block 1008 to block 1012, where the computing system aligns the plurality of sequence reads to a sequence graph to generate a plurality of aligned sequence reads. The sequence graph can represent a locus of a gene of interest (e.g., RFC1). The sequence graph can comprise a repeat sequence representation (e.g., AARRG where R is A or G if the gene of interest is RFC1) flanked by non-repeat sequences of the locus of the gene. The plurality of aligned sequence reads can comprise the plurality of sequence reads. The plurality of aligned sequence reads can comprise or be associated with alignments of the plurality of sequence reads to the sequence graph.

The method 1000 proceeds from block 1012 to block 1016, where the computing system determines a number of occurrences of a plurality of repeat sequences (e.g., AAGGG, AAAAG, AAAGG, AAGAG, AACGG, and ACGGG if the gene of interest is RFC1) in aligned sequence reads of the plurality of aligned sequence reads using a first occurrence threshold (e.g., 2) and a first quality threshold (e.g., 20). Determining or counting the number of occurrences of the plurality of repeat sequences in the aligned sequence reads of the plurality of aligned sequence reads using the first occurrence threshold and the first quality threshold is referred to herein as n-mer filtering and counting, where n is the length of the repeat sequence. The n-mer filtering and counting can be for both alleles or for the expanded allele.

A repeat expansion at the locus of the gene of interest can comprise greater than a threshold total copies of one or more repeat sequences. The threshold total copies of one or more repeat sequences can be, for example, 15, 16, 17, 18, 19, 20, 30, 40, 50, 60, 70, 80, 90, 100, or more or less, total copies of one or more repeat sequences.

In some embodiments, each of the plurality of repeat sequences has a number of occurrences greater than or equal to (or greater than) a first occurrence threshold with each occurrence having a number of bases each having a quality score greater than or equal to (or greater than) a first quality threshold. The first occurrence threshold can be, for example, 2, 3, 4, 5, 6, 7, 8, 9, 10, or more. The first quality threshold can be, for example, about 10, 15, 20, 25, 30, 35, 40, 45, 50, or more or less. The number of bases of the repeat sequence each having a quality score greater than (or greater than or equal to) the first quality threshold can be, for example, 4, 5, 6, 7, 9, 9, 10, or more. For example, each type of 5-mer has two occurrences with all 5 bases having quality (Q) greater than or equal to a quality threshold (e.g., a first quality threshold) of 20. In some embodiments, each of the occurrences has a number of bases each having a quality score greater than or equal to (or greater than) than a second quality threshold. The number of bases each having a quality score greater than or equal to (or greater than) than the second quality threshold can be, for example, 2, 3, 4, 5, 6, 7, 8, 9, 10, or more. The second quality threshold can be for example, about 10, 15, 20, 25, 30, 35, 40, 45, 50, or more or less. The number of bases of each of the occurrences having a quality score greater than the second quality threshold can be, for example, 2, 3, 4, or 5. For example, each counted 5-mer has at least 3 bases having quality greater than or equal to a quality threshold (e.g., a second quality threshold) of 20. In some embodiments, the first quality threshold and the second quality threshold are identical.

The repeat sequence representation can be degenerate. The repeat sequence representation can be AARRG, where R is A or G, if the gene of insert is RFC1. The repeat sequence representation can be 5, 6, 7, 8, 9, 10, or more, bases in length. Each of the plurality of repeat sequences can be 5, 6, 7, 8, 9, 10, or more, bases in length. The repeat sequence representation and a repeat sequence can have an identical length.

The method 1000 proceeds from block 1016 to block 1020, where the computing system determines a frequency indication of a number of occurrences of a pathogenic repeat sequence (e.g., AAGGG or ACAGG if the gene of interest is RFC1), or one or more pathogenic repeat sequences, relative to a total number of occurrences of the plurality of repeat sequence. The pathogenic repeat sequence can have a GC content of at least (or greater than) 50%, 55%, 60%, 65%, 70%, 75%, 80%, or more or less. The pathogenic repeat sequence can be 5, 6, 7, 8, 9, 10, or more, bases in length.

The frequency indication of the number of occurrences of the pathogenic repeat sequence relative to the total number of occurrences of the plurality of repeat sequences can be a percentage of the number of occurrences of the pathogenic repeat sequence out of the total number of occurrences of the plurality of repeat sequences. The frequency indication of the number of occurrences of the pathogenic repeat sequence relative to the total number of occurrences of the plurality of repeat sequences can be a ratio of the number of occurrences of the pathogenic repeat sequence over the total number of occurrences of the plurality of repeat sequences.

The method 1000 proceeds from block 1020 to block 1024, where the computing system determines a repeat expansion status at the locus of the gene of interest of the subject using the frequency indication of the number of occurrences of the pathogenic repeat sequence relative to the total number of occurrences of the plurality of repeat sequences. The computing system can determine the subject has zero, one, or two alleles with a repeat expansion at the locus of the gene of interest using the plurality of aligned sequence reads. The repeat expansion status at the locus of the gene of interest is a pathogenic status, a carrier status, or a benign status. The repeat expansion can be associated with or causes a disease. The disease can be, for example, late-onset ataxia, cerebellar ataxia , sensory neuronopathy, bilateral vestibulopathy, or cerebellar ataxia, neuropathy, and vestibular areflexia syndrome (CANVAS). The disease can be a cancer, a non-cancer disease, a neurological disease, a neurodegenerative disease, an autoimmune disease, Alzheimer’s disease, Parkinson’s Disease, dementia, rheumatoid arthritis, or inflammation. The disease can be Huntington disease, spinal and bulbar muscular atrophy, dentatorubral-pallidoluysian atrophy spinocerebellar ataxias, fragile X, fragile X tremor ataxia syndrome, other fragile sites, myotonic dystrophy type 1, Huntington disease-like 2, spinocerebellar ataxia type 8, Fuchs corneal dystrophy, Friedreich ataxia, FRAXE mental retardation, oculopharyngeal muscular dystrophy, myotonic dystrophy type 1, spinocerebellar ataxia type 10, spinocerebellar ataxia type 31, spinocerebellar ataxia type 36, frontotemporal dementia/amyotrophic lateral sclerosis, or EPM1 (myoclonic epilepsy). The gene of interest can be Huntingtin, androgen receptor (AR) gene, ATN1, ATXN1, ATXN2, ATXN3, ATXN10, CACNA1A, ATXN7, TBP gene, PPP2R2B, TK2, BEAN, NOP56, JPH3, FRDA, CSTB, PABP2, TCF4, or C9ORF72.

In some embodiments, the gene of interest is replication factor C subunit 1 (RFC1). The locus of the gene of interest with a repeat expansion can be at, or at about, chr4:39348424 of hg38, or a corresponding position of another reference genome sequence. The repeat expansion can be associated with or causes a disease. The disease can be cerebellar ataxia, neuropathy, and vestibular areflexia syndrome (CANVAS) or ataxia. The repeat sequence representation can be AARRG. The pathogenic repeat sequence can be AAGGG or ACAGG.

Two expanded alleles. If both alleles are expanded, 5-mer counting of both expanded alleles can be performed. In some embodiments, the subject can have two alleles each with a repeat expansion at the locus of the gene of interest.

Two expanded alleles - Pathogenic status. To determine the repeat expansion status at the locus of the gene of interest, the computing system can determine the frequency indication of the number of occurrences of the pathogenic repeat sequence relative to the total number of occurrences of the plurality of repeat sequences is greater than (or greater than or equal to) a first status threshold. The first status threshold can be, for example, 60%, 65%, 70%, 75%, 80%, 85%, 90%, 95%, or more or less. The computing system can determine the repeat expansion status at the locus of the gene of interest as the pathogenic status.

Two expanded alleles - Carrier status. To determine the repeat expansion status at the locus of the gene of interest, the computing system can determine the frequency indication of the number of occurrences of the pathogenic repeat sequence relative to the total number of occurrences of the plurality of repeat sequences is less than or equal to (or less than) a first status threshold and greater than or equal to (or greater than) a second status threshold. The first status threshold can be, for example, 60%, 65%, 70%, 75%, 80%, 85%, 90%, 95%, or more or less. The second status threshold can be, for example, 10%, 15%, 20%, 25%, 30%, 35%, 40%, 45%, 50%, or more or less. The computing system can determine the repeat expansion status at the locus of the gene of interest as the carrier status.

Two expanded alleles - Carrier status. To determine the repeat expansion status at the locus of the gene of interest, the computing system can determine the frequency indication of the number of occurrences of the pathogenic repeat sequence relative to the total number of occurrences of the plurality of repeat sequences is less than or equal to (or less than) a first status threshold and is greater than or equal to (or is greater than or equal to) a second status threshold. The first status threshold can be, for example, 60%, 65%, 70%, 75%, 80%, 85%, 90%, 95%, or more or less. The second status threshold can be, for example, 10%, 15%, 20%, 25%, 30%, 35%, 40%, 45%, 50%, or more or less.

The computing system can determine a frequency indication of a number of occurrences of (1) the pathogenic repeat sequence and (2) a sequence with a high sequence similarity to the pathogenic repeat sequence relative to the total number of occurrences of the plurality of repeat sequences. The sequence similarity of the pathogenic repeat sequence and the sequence with a high sequence similarity to the pathogenic repeat sequence can be, or be about, for example, 70%, 75%, 80%, 85%, 90%, 95%, or more or less. The pathogenic repeat sequence and the sequence similar to the pathogenic repeat sequence differs by one or more bases, such as 1, 2, 3, 4, 5, 6, 7, 8, 9, 10, or more, bases. The sequence with a high sequence similarity to the pathogenic repeat sequence may be non-pathogenic and/or associated with (e.g., linked with) benign expansion.

The frequency indication of the number of occurrences of (1) the pathogenic repeat sequence and (2) the sequence with a high sequence similarity to the pathogenic repeat sequence, relative to the total number of occurrences of the plurality of repeat sequences, can be a percentage of the number of occurrences of (1) the pathogenic repeat sequence and (2) the sequence with a high sequence similarity to the pathogenic repeat sequence out of the total number of occurrences of the plurality of repeat sequences. The frequency indication of the number of occurrences of (1) the pathogenic repeat sequence and (2) the sequence with a high sequence similarity to the pathogenic repeat sequence, relative to the total number of occurrences of the plurality of repeat sequences, can be a ratio of the number of occurrences of (1) the pathogenic repeat sequence and (2) the sequence with a high sequence similarity to the pathogenic repeat sequence over the total number of occurrences of the plurality of repeat sequences.

The computing system can determine frequency indication of a number of occurrences of the pathogenic repeat sequence and the sequence with a high sequence similarity to the pathogenic repeat sequence relative to the total number of occurrences of the plurality of repeat sequences is greater than (or greater than or equal to) a third status threshold. The third status threshold can be, for example, 70%, 75%, 80%, 85%, 90%, 95%, or more or less.

The computing system can determine the frequency indication of the number of occurrences of the pathogenic sequence is greater than (or greater than or equal to) a fourth status threshold. The fourth status threshold can be, for example, 5%, 10%, 15%, 20%, 25%, 30%, or more or less. Alternatively or additionally, the computing system can determine a frequency indication of a number of occurrences of the sequence with a high sequence similarity to the pathogenic sequence. The frequency indication of number of occurrences of the sequence with a high sequence similarity to the pathogenic repeat sequence, relative to the total number of occurrences of the plurality of repeat sequences, can be a percentage of the number of occurrences of the sequence with a high sequence similarity to the pathogenic repeat sequence out of the total number of occurrences of the plurality of repeat sequences. The frequency indication of the sequence with a high sequence similarity to the pathogenic repeat sequence, relative to the total number of occurrences of the plurality of repeat sequences, can be a ratio of the number of occurrences of the sequence with a high sequence similarity to the pathogenic repeat sequence over the total number of occurrences of the plurality of repeat sequences. The computing system can determine the frequency indication of the number of occurrences of the sequence with a high sequence similarity to the pathogenic sequence is less than or equal to (or less than) a fifth status threshold. The fifth status threshold can be, for example, 70%, 75%, 80%, 85%, 90%, 95%, or more or less. The computing system can determine the repeat expansion status at the locus of the gene of interest as the carrier status.

In some embodiments, the computing system determines the frequency indication of the number of occurrences of the pathogenic sequence is less than or equal to (or less than) the fourth status threshold. Alternatively or additionally, the computing system can determine the frequency indication of the number of occurrences of the sequence with a high sequence similarity to the pathogenic sequence is greater than (or greater than or equal to) the fifth status threshold. The computing system can determine the repeat expansion status at the locus of RFC1 as the benign status.

Two expanded alleles - Benign status. To determine the repeat expansion status at the locus of the gene of interest, the computing system can determine the frequency indication of the number of occurrences of the pathogenic repeat sequence relative to the total number of occurrences of the plurality of repeat sequences is less than (or less than or equal to) a second status threshold. The second status threshold can be, for example, 10%, 15%, 20%, 25%, 30%, 35%, 40%, 45%, 50%, or more or less. The computing system can determine the repeat expansion status at the locus of the gene of interest as the benign status.

2. One expanded allele. If one allele is expanded and one allele is not expanded, 5-mer counting of the expanded allele can be performed. In some embodiments, the subject has one allele of the gene of interest with repeat expansion at the locus of the gene of interest.

One expanded allele - Pathogenic status. To determine the number of occurrences of the plurality of repeat sequences in the aligned sequence reads of the plurality of sequence reads, the computing system can determine the number of occurrences of the plurality of repeat sequences in the aligned sequence reads of the plurality of aligned sequence reads that are from the one allele of the gene of interest with repeat expansion at the locus of RFC1. To determine the repeat expansion status at the locus of the gene of interest, the computing system can determine the frequency indication of the number of occurrences of the pathogenic repeat sequence relative to the total number of occurrences of the plurality of repeat sequences is greater than (or greater than or equal to) a first status threshold. The first status threshold can be, for example, 60%, 65%, 70%, 75%, 80%, 85%, 90%, 95%, or more or less. The computing system can determine the repeat expansion status at the locus of the gene of interest as the pathogenic status.

One expanded allele - Carrier status. To determine the number of occurrences of the plurality of repeat sequences in the aligned sequence reads of the plurality of sequence reads, the computing system can determine the number of occurrences of the plurality of repeat sequences in the aligned sequence reads of the plurality of aligned sequence reads that are from the one allele of the gene of interest with repeat expansion at the locus of RFC1. To determine the repeat expansion status at the locus of the gene of interest, the computing system can determine the frequency indication of the number of occurrences of the pathogenic repeat sequence relative to the total number of occurrences of the plurality of repeat sequences is less than or equal to (or less than) a first status threshold. The first status threshold can be, for example, 60%, 65%, 70%, 75%, 80%, 85%, 90%, 95%, or more or less. The computing system can determine the repeat expansion status at the locus of the gene of interest as the carrier status.

To determine the repeat expansion status at the locus of the gene of interest as the pathogenic status or the carrier status when there is one expanded allele, the computing system can select the aligned sequence reads that are from the one allele of the gene of interest with repeat expansion at the locus of the gene of interest based on the alignments of the aligned sequence reads. The aligned sequence reads that are from the one allele of the gene of interest with repeat expansion at the locus of the gene of interest can comprise the aligned sequence reads that are (i) in-repeat reads or (ii) flanking reads each with an overlap to the repeat expansion at the locus of the gene of interest greater than a repeat expansion overlap threshold. The repeat expansion overlap threshold can be, for example, 30, 35, 40, 45, 50, 55, 60, 65, 70, 75, 80, 85, 90, or more or less, base pairs. The aligned sequence reads that are from the one allele of the gene of interest with repeat expansion at the locus of the gene of interest can comprise (i) all the aligned sequence reads that are in-repeat reads and (ii) not all of the aligned sequence reads that are flanking reads. An in-repeat read can occur when the repeat is longer than the read length such that the entire read (whether a single-end sequencing read or a pair-end sequencing read) or one read of a pair-end sequencing read includes only a portion of the repeat and no flanking region of the repeat. A flanking read includes a portion of the repeat and the flanking region on one end of the repeat.

No expanded allele. If both alleles are not expanded, 5-mer counting of both alleles is can be performed. In some embodiments, the subject has zero allele with repeat expansion at the locus of the gene of interest. The repeat expansion status at the locus of the gene of interest can be benign status.

In some embodiments, the computing system can generate a user interface (UI), such as a graphical user interface, comprising or representing any results (including intermediate results) of the method 1000. For example, the UI can comprise or represent the repeat expansion status. The UI can include, for example, a dashboard. The UI can include one or more UI elements. A UI element can comprise or represent the repeat expansion status. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion). In some embodiments, the computing system can generate a report comprising or representing any results (including intermediate results) of the method 900, such as the repeat expansion status.

The computing system can cause one or more diagnosis methods to be performed to confirm of the repeat expansion status at the locus of the gene of interest of the subject. The UI or the report can comprise or indicate that one or more diagnosis methods should be performed to confirm the repeat expansion status at the locus of the gene of interest of the subject. The computing system can receive conformation of the repeat expansion status at the locus of the gene of interest of the subject determined using one or more diagnosis systems. The one or more diagnosis systems can comprise polymerase chain reaction (PCR) and Sanger sequencing, southern blots, and linkage analysis.

In some embodiments, any threshold of the method 1000 (such as an occurrence threshold (e.g., a first occurrence threshold), a quality threshold (e.g., a first quality threshold, or a second quality threshold), a threshold total copies, a status threshold (e.g., a first status threshold, a second status threshold, a third status threshold, a fourth status threshold, or a fifth status threshold), or a repeat expansion overlap threshold) can be determined using a number of samples, such as 100, 200, 300, 400, 500, 1000, 2000, 3000, 4000, 5000, or more or less, samples.

The method 1000 ends at block 1028.

### Execution Environment

FIG. 11 depicts a general architecture of an example computing device 1100 configured for determining repeat expansion status of a gene of interest (e.g., RFC1). The general architecture of the computing device 1100 depicted in FIG. 11 includes an arrangement of computer hardware and software components. The computing device 1100 may include many more (or fewer) elements than those shown in FIG. 11. It is not necessary, however, that all of these generally conventional elements be shown in order to provide an enabling disclosure. As illustrated, the computing device 1100 includes a processing unit 1110, a network interface 1120, a computer readable medium drive 1130, an input/output device interface 1140, a display 1150, and an input device 1160, all of which may communicate with one another by way of a communication bus. The network interface 1120 may provide connectivity to one or more networks or computing systems. The processing unit 1110 may thus receive information and instructions from other computing systems or services via a network. The processing unit 1110 may also communicate to and from memory 1170 and further provide output information for an optional display 1150 via the input/output device interface 1140. The input/output device interface 1140 may also accept input from the optional input device 1160, such as a keyboard, mouse, digital pen, microphone, touch screen, gesture recognition system, voice recognition system, gamepad, accelerometer, gyroscope, or other input device.

The memory 1170 may contain computer program instructions (grouped as modules or components in some embodiments) that the processing unit 1110 executes in order to implement one or more embodiments. The memory 1170 generally includes RAM, ROM and/or other persistent, auxiliary or non-transitory computer-readable media. The memory 1170 may store an operating system 1172 that provides computer program instructions for use by the processing unit 1110 in the general administration and operation of the computing device 1100. The memory 1170 may further include computer program instructions and other information for implementing aspects of the present disclosure.

For example, in one embodiment, the memory 1170 includes a repeat expansion status determining module 1174 for determining repeat expansion status (e.g., pathogenic, carrier, or benign), such as the method 900 described with reference to FIG. 9 and the method 1000 described with reference to FIG. 10. In addition, memory 1170 may include or communicate with the data store 1190 and/or one or more other data stores that store sequence reads being processed and repeat expansion status determined (and any intermediate results thereof).

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described greater than without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

1-39. (canceled)

40. A system for determining a repeat expansion status of a gene of interest comprising:
non-transitory memory configured to store executable instructions; and
a hardware processor in communication with the non-transitory memory, the hardware processor programmed by the executable instructions to perform:
(a) receiving a plurality of sequence reads generated from a sample obtained from a subject;
(b) aligning the plurality of sequence reads to a sequence graph to generate a plurality of aligned sequence reads, wherein the sequence graph represents a locus of a gene of interest and comprises a repeat sequence representation flanked by non-repeat sequences of the locus of the gene, and wherein the plurality of aligned sequence reads comprises the plurality of sequence reads and alignments of the plurality of sequence reads to the sequence graph;
(c) determining a number of occurrences of a plurality of repeat sequences in aligned sequence reads of the plurality of aligned sequence reads using a first occurrence threshold and a first quality threshold;
(d) determining a frequency indication of a number of occurrences of a pathogenic repeat sequence relative to a total number of occurrences of the plurality of repeat sequence(e) determining a repeat expansion status at the locus of the gene of interest of the subject using the frequency indication of the number of occurrences of the pathogenic repeat sequence relative to the total number of occurrences of the plurality of repeat sequences.

41. The system of claim 40, wherein the gene of interest is replication factor C subunit 1 (RFC1).

42. (canceled)

43. The system of claim 41 , wherein the repeat expansion is associated with or causes a disease, optionally wherein the disease is cerebellar ataxia, neuropathy, and vestibular areflexia syndrome (CANVAS).

44. The system of claim 41, wherein the repeat sequence representation is AARRG.

45. The system of claim 41, wherein the pathogenic repeat sequence is AAGGG or ACAGG.

46. The system of claim, wherein the hardware processor is programmed by the executable instructions to perform: determining the subject has zero, one, or two alleles with a repeat expansion at the locus of the gene of interest using the plurality of aligned sequence reads.

47. The system of claim, wherein the subject has two alleles with repeat expansion at the locus of the gene of interest.

48. The system of claim 47, wherein determining the repeat expansion status at the locus of the gene of interest comprises:
determining the frequency indication of the number of occurrences of the pathogenic repeat sequence relative to the total number of occurrences of the plurality of repeat sequences is greater than a first status threshold; and
determining the repeat expansion status at the locus of the gene of interest as pathogenic status.

49-51. (canceled)

52. The system of claim, wherein the subject has one allele of the gene of interest with repeat expansion at the locus of the gene of interest.

53-57. (canceled)

58. The system of claim, wherein the subject has zero allele with repeat expansion at the locus of the gene of interest, and wherein the repeat expansion status at the locus of the gene of interest is benign status.

59-61. (canceled)

62. The system of claim, wherein the repeat expansion status at the locus of the gene of interest is a pathogenic status, a carrier status, or a benign status.

63. The system of claim , wherein each of the plurality of repeat sequences has a number of occurrences greater than or equal to the first occurrence threshold with each occurrence having a number of bases each having a quality score greater than or equal to the first quality threshold.

64. (canceled)

65. The system of claim, wherein each of the occurrences has a number of bases each having a quality score greater than or equal to a second quality threshold.

66. (canceled)

67. The system of claim 40, wherein the repeat sequence representation is degenerate.

68. (canceled)

69. The system of claim 40, wherein the repeat sequence representation and/or each of the plurality of repeat sequences is at least 5 bases in length, optionally wherein the repeat sequence representation and/or each of the plurality of repeat sequences is 6 bases in length.

70. The system of claim 40, wherein the pathogenic repeat sequence has a GC content of at least 60%.

71. The system of claim 40, wherein the frequency indication of the number of occurrences of the pathogenic repeat sequence relative to the total number of occurrences of the plurality of repeat sequences is a percentage of the number of occurrences of the pathogenic repeat sequence out of the total number of occurrences of the plurality of repeat sequences or a ratio of the number of occurrences of the pathogenic repeat sequence over the total number of occurrences of the plurality of repeat sequences.

72. The system of claim 40, wherein the plurality of sequence reads is aligned to the locus of the gene of interest.

73. The system of claim 40, wherein receiving the plurality of sequence reads generated from the sample obtained comprises:
aligning a second plurality of sequence reads comprising the plurality of sequence reads to a reference genome sequence; and
selecting the plurality of sequence reads from the second plurality of sequence reads, wherein the plurality of sequence reads is aligned to the locus of the gene of interest.

74-79. (canceled)

80. The system of claim 40 , wherein the hardware processor is programmed by the executable instructions to perform: receiving conformation of the repeat expansion status at the locus of the gene of interest of the subject determined using one or more diagnosis systems, optionally wherein the one or more diagnosis systems comprise polymerase chain reaction (PCR) and Sanger sequencing, southern blots, and linkage analysis.


SEQUENCE LISTING (46 sequences)

SEQ ID NO: 1 - DNA, 51 bp, Homo Sapiens
gaagggaagggaagggaagggaagggaagggaagggaagggaagggaaggg

SEQ ID NO: 2 - DNA, 54 bp, Homo Sapiens
agggaagggaagggaagggaagggaagggaagggaagggaagggcagggcaggg

SEQ ID NO: 3 - DNA, 54 bp, Homo Sapiens
agggaagggaagggaagggaagggaagggaagggaagggaagggaagggaaggg

SEQ ID NO: 4 - DNA, 55 bp, Homo Sapiens
aagggaagggaagggaagggaagggaagggaagggaagggaagggaagggaaggg

SEQ ID NO: 5 - DNA, 55 bp, Homo Sapiens; identical to SEQ ID NO: 4

SEQ ID NO: 6 - DNA, 55 bp, Homo Sapiens; identical to SEQ ID NO: 4

SEQ ID NO: 7 - DNA, 55 bp, Homo Sapiens; identical to SEQ ID NO: 4

SEQ ID NO: 8 - DNA, 55 bp, Homo Sapiens; identical to SEQ ID NO: 4

SEQ ID NO: 9 - DNA, 55 bp, Homo Sapiens
  misc_feature LOCATION: (6)..(6): n is a, c, g, or t
  misc_feature LOCATION: (12)..(12): n is a, c, g, or t
  misc_feature LOCATION: (26)..(26): n is a, c, g, or t
  misc_feature LOCATION: (41)..(42): n is a, c, g, or t
  misc_feature LOCATION: (52)..(52): n is a, c, g, or t
acgggnggggcngggcaggggggggnggggtgggggggggnngggagggggnggg

SEQ ID NO: 10 - DNA, 55 bp, Homo Sapiens
gggggaggggggggggggggggggggggggggggggggggggggggagggggggg

SEQ ID NO: 11 - DNA, 55 bp, Homo Sapiens
  misc_feature LOCATION: (12)..(12): n is a, c, g, or t
  misc_feature LOCATION: (37)..(37): n is a, c, g, or t
  misc_feature LOCATION: (46)..(47): n is a, c, g, or t
gagggggaggangggagagggagggaagagggggggngggaggagnngggaaggg

SEQ ID NO: 12 - DNA, 40 bp, Homo Sapiens
  misc_feature LOCATION: (27)..(27): n is a, c, g, or t
  misc_feature LOCATION: (30)..(30): n is a, c, g, or t
  misc_feature LOCATION: (32)..(32): n is a, c, g, or t
aaaaaaaaagaaaagaaaagaaaaganganangagaagag

SEQ ID NO: 13 - DNA, 40 bp, Homo Sapiens
aagggaagggaagggaagggaagggaaggggagggagggg

SEQ ID NO: 14 - DNA, 40 bp, Homo Sapiens
aagggaagggaagggaagggaagggaagggaagggaaggg

SEQ ID NO: 15 - DNA, 40 bp, Homo Sapiens; identical to SEQ ID NO: 14

SEQ ID NO: 16 - DNA, 40 bp, Homo Sapiens
  misc_feature LOCATION: (15)..(15): n is a, c, g, or t
  misc_feature LOCATION: (21)..(21): n is a, c, g, or t
  misc_feature LOCATION: (23)..(23): n is a, c, g, or t
  misc_feature LOCATION: (30)..(30): n is a, c, g, or t
  misc_feature LOCATION: (35)..(35): n is a, c, g, or t
aaaaaaaaaaaaaanaaaagnanagaaaanaaaanaagag

SEQ ID NO: 17 - DNA, 40 bp, Homo Sapiens; identical to SEQ ID NO: 14

SEQ ID NO: 18 - DNA, 40 bp, Homo Sapiens
aagagaagagaagagaagagaagagaagagaagagaagag

SEQ ID NO: 19 - DNA, 40 bp, Homo Sapiens
  misc_feature LOCATION: (2)..(2): n is a, c, g, or t
  misc_feature LOCATION: (6)..(7): n is a, c, g, or t
gngggnngggaaggggggagaggagaagggtggggagggg

SEQ ID NO: 20 - DNA, 40 bp, Homo Sapiens
  misc_feature LOCATION: (15)..(15): n is a, c, g, or t
  misc_feature LOCATION: (22)..(22): n is a, c, g, or t
  misc_feature LOCATION: (27)..(27): n is a, c, g, or t
  misc_feature LOCATION: (37)..(37): n is a, c, g, or t
aggggggggggaggnaggggangggangagaagaganggg

SEQ ID NO: 21 - DNA, 40 bp, Homo Sapiens; identical to SEQ ID NO: 14

SEQ ID NO: 22 - DNA, 40 bp, Homo Sapiens
  misc_feature LOCATION: (2)..(2): n is a, c, g, or t
  misc_feature LOCATION: (26)..(26): n is a, c, g, or t
  misc_feature LOCATION: (37)..(37): n is a, c, g, or t
anggcacgggggagggtgacacacgnggggaggggtnggg

SEQ ID NO: 23 - DNA, 40 bp, Homo Sapiens
aagaggagggaagagaggaggagagaagggaagagaagag

SEQ ID NO: 24 - DNA, 40 bp, Homo Sapiens
aagagaaaaaaagagaaaagaagaaaagagaagaaaagag

SEQ ID NO: 25 - DNA, 40 bp, Homo Sapiens
  misc_feature LOCATION: (4)..(5): n is a, c, g, or t
  misc_feature LOCATION: (10)..(10): n is a, c, g, or t
aaannaaaanaagagaagagaagagaagagaagagaagag

SEQ ID NO: 26 - DNA, 40 bp, Homo Sapiens
  misc_feature LOCATION: (20)..(20): n is a, c, g, or t
  misc_feature LOCATION: (22)..(22): n is a, c, g, or t
  misc_feature LOCATION: (24)..(24): n is a, c, g, or t
aagagaaagggagagaaagnanangaaaagaagagaagaa

SEQ ID NO: 27 - DNA, 40 bp, Homo Sapiens
  misc_feature LOCATION: (16)..(16): n is a, c, g, or t
  misc_feature LOCATION: (31)..(32): n is a, c, g, or t
tggggaggggaagagnggggaaggggggggnngggagggg

SEQ ID NO: 28 - DNA, 40 bp, Homo Sapiens; identical to SEQ ID NO: 14

SEQ ID NO: 29 - DNA, 40 bp, Homo Sapiens; identical to SEQ ID NO: 14

SEQ ID NO: 30 - DNA, 40 bp, Homo Sapiens; identical to SEQ ID NO: 14

SEQ ID NO: 31 - DNA, 40 bp, Homo Sapiens; identical to SEQ ID NO: 14

SEQ ID NO: 32 - DNA, 40 bp, Homo Sapiens; identical to SEQ ID NO: 14

SEQ ID NO: 33 - DNA, 40 bp, Homo Sapiens
  misc_feature LOCATION: (6)..(7): n is a, c, g, or t
aggggnngggaaggggaaggaagggaagggaagggaaggg

SEQ ID NO: 34 - DNA, 40 bp, Homo Sapiens; identical to SEQ ID NO: 14

SEQ ID NO: 35 - DNA, 40 bp, Homo Sapiens
  misc_feature LOCATION: (15)..(17): n is a, c, g, or t
aagggggggggtggnnngggaagggaagggtcgggaaggg

SEQ ID NO: 36 - DNA, 40 bp, Homo Sapiens
cagggacgagaggggaagggacggggccgggagggcgggg

SEQ ID NO: 37 - DNA, 40 bp, Homo Sapiens; identical to SEQ ID NO: 14

SEQ ID NO: 38 - DNA, 40 bp, Homo Sapiens
aaaggaatgggatagaaaggataggaagggagaggagggg

SEQ ID NO: 39 - DNA, 40 bp, Homo Sapiens
aggggaggggaagggaagggaagggaagcgaagggaaggg

SEQ ID NO: 40 - DNA, 30 bp, Homo Sapiens
aaaagaaaagaaaagaaaagaaaagaaaag

SEQ ID NO: 41 - DNA, 30 bp, Homo Sapiens
gaaagaaaagaatagaaaggaaaagataag

SEQ ID NO: 42 - DNA, 30 bp, Homo Sapiens; identical to SEQ ID NO: 40

SEQ ID NO: 43 - DNA, 30 bp, Homo Sapiens; identical to SEQ ID NO: 40

SEQ ID NO: 44 - DNA, 30 bp, Homo Sapiens; identical to SEQ ID NO: 40

SEQ ID NO: 45 - DNA, 30 bp, Homo Sapiens; identical to SEQ ID NO: 40

SEQ ID NO: 46 - DNA, 30 bp, Homo Sapiens; identical to SEQ ID NO: 40